(19) United States
(12) Patent Application Publication
(10) Pub. No.: US 2020/0038166 A1
(43) Pub. Date: Feb. 6, 2020

(54) DEPLOYABLE AND RETRACTABLE ANCHOR

(71) Applicant: Cook Medical Technologies LLC, Bloomington, IN (US)

(72) Inventor: Woong KIM, West Lafayette, IN (US)

(73) Assignee: Cook Medical Technologies LLC

(21) Appl. No.: 16/530,098

(22) Filed: Aug. 2, 2019

Related U.S. Application Data
(60) Provisional application No. 62/714,209, filed on Aug. 3, 2018.
(60) Provisional application No. 62/714,212, filed on Aug. 3, 2018.
(60) Provisional application No. 62/714,213, filed on Aug. 3, 2018.

(51) Int. Cl. A61F 2/07; A61F 2/82

(52) U.S. Cl. CPC A61F 2/07; A61L 31/14; A61F 2002/075; A61F 2/82

(57) ABSTRACT

Disclosed herein is an anchor for use with a medical device. The anchor may be cut from a portion of the body of the medical device, such as a strut, and may have a remembered state which is a collapsed state, and an expanded state. The movable anchor renders the device repositionable such that it may be implanted with great precision. The medical device may be a stent or a stent graft.

### CROSS REFERENCE TO RELATED APPLICATIONS

This application claims priority to U.S. Provisional Application No. 62/714,209, entitled “Deployable and Retractable Anchor,” filed on Aug. 3, 2018; U.S. Provisional Application No. 62/714,212, entitled “Proximal-Distal Steerable Stent with Length Control Capability,” filed on Aug. 3, 2018; and U.S. Provisional Application No. 62/714,213, entitled “Delivery System That Can Control Stent Radius Bidirectionally During Deployment,” filed on Aug. 3, 2018; the entire contents of all of which are incorporated by reference herein.

### BACKGROUND

The present application generally relates to medical devices. More particularly, the present application relates to an anchor for an intravascular implant, the implantable device itself, and methods of making and using the same.

Medical devices that are permanently or temporarily deployed into the lumen of a body vessel are generally placed with precision, into areas of the body that require treatment. These devices may perform ideally when they remain at the site to which they were deployed. In order to retain these devices in such locations, a number of fixation methods have been developed.

In some devices, barbs or anchors that protrude radially away from the device body and engage tissue are utilized. These barbs or anchors may taper to a sharpened tip or point in order to embed a portion of the device into the wall of the body vessel to which they are deployed in order to prevent movement of the device.

Although effective, known anchors and barbs can be improved upon. The static nature of known barbs may have a number of drawbacks. First, since the barbs tend to be the outermost portions of the device, known barbs can increase the profile of a device in which they are employed. Additionally, because the barbs attach firmly to the tissue that they contact, the device tends to remain at the position in which the barbs initially contact the tissue. This in turn may cause physicians to be reluctant to move the device as the risk of tearing tissue can outweigh the benefit of adjusting positioning.

A particular type of medical implant that has been used with barbs to anchor the device in place is a stent, either in its bare form, or as a portion of a stent graft. In some instances, stents are made of shape memory materials, but despite the shape memory properties of the materials of these devices, the barbs remain static. Moreover, such barbs can introduce unpredictability into the implantation procedure, as a spring-back force from the stent increases the difficulty of predicting the exact orientation of the device during the final release of the stent from the delivery system.

Moreover, stent grafts function in conjunction with a contractual reaction force generated by the wall of the vessel to which they are deployed to provide both fixing and sealing of the device. Therefore, the shape, size, and integrity of the vessel at the landing zone may play a role in the efficacy of endovascular repair. Ideally, the landing zone will be a symmetric, relatively straight and cylindrical shape, having a length which permits implantation (such as about 15 mm or longer). When morphology of an ideal vessel meets these criteria, the outer surface of the deployed device would mate well with the inner wall of the vessel, without gaps, while the anchors would be entrenched evenly in the tissue layer for secure fixation. The device would then divert hemodynamic pressure into the device and away from the vessel wall, operating indefinitely without migration or endoleak. However, in reality, as disease progresses, the chances of anatomical changes increase, and it becomes more difficult to identify a suitable landing zone as the disease progresses.

It has been a challenge to develop an anchor for a medical device which allows for repositioning during deployment and implantation, and which has the ability to generate a landing zone suitable for deployment in a diseased vessel.

### SUMMARY

In one aspect, the present disclosure provides a medical device for implantation into a body lumen. The medical device may be movable between a delivery configuration and an expanded configuration. The medical device may include an anchor including an elongate ribbon extending from a first end to a second end and a longitudinally expandable section defining at least one curve. The first end of the elongate ribbon may be connected to the longitudinally expandable section. The elongate ribbon may include a barb. The medical device may define a lumen. The anchor may be disposed within the lumen in the delivery configuration. The anchor may extend outward radially from the lumen in the expanded configuration.

In another aspect, the present disclosure provides a stent for implantation into a body lumen. The stent may include a tubular body defining a lumen therein and longitudinal axis therethrough. The stent may be movable between a delivery configuration and an expanded configuration. The stent may include an anchor including an elongate ribbon extending from a first end to a second end and a longitudinally expandable section defining at least one curve. The first end of the elongate ribbon may be connected to the longitudinally expandable section. The anchor may be disposed within the lumen when the stent is in the delivery configuration. The anchor may extend outward radially from the lumen in the expanded configuration.

In another aspect, the present disclosure provides a method of making a stent. The method may include a step of cutting a shape memory metal cannula to define a stent body, the stent body being tubular and defining a lumen therethrough, the stent body including at least one anchor for contacting tissue, the anchor comprising a longitudinally expandable section and an elongate ribbon extending from a first end to a second end, the first end of the elongate ribbon being connected to the longitudinally expandable section, the elongate ribbon comprising a barb. The method may include a step of placing the stent body in a mandrel, the mandrel defining a first curved surface and a second curved surface, such that the anchor is disposed between the first curved surface and the second curved surface, and within an interior of the stent body. The method may include a step of heating the stent body to a temperature effective to define a remembered state wherein the anchor is disposed within the interior of the stent body.

In another aspect, the present disclosure provides a medical device for implantation into a body lumen, the medical device being movable between a delivery configuration and an expanded configuration. The medical device may include an anchor comprising an elongate ribbon extending from a first end to a second end and a longitudinally expandable section defining at least one curve. The first end of the elongate ribbon may be connected to the longitudinally expandable section. The elongate ribbon may include a barb formed therefrom. The medical device may include at least one aperture for receiving a release wire therethrough. The medical device may define an outer perimeter, the anchor being restrained within the outer perimeter by the release wire in the delivery configuration, and the anchor extending outward radially from the outer perimeter in the expanded configuration.

In another aspect, the present disclosure provides a stent for implantation into a body lumen. The stent may include a tubular body defining a lumen therethrough and a longitudinal axis. The stent may be movable between a delivery configuration and an expanded configuration. The stent may include an anchor including an elongate ribbon extending from a first end to a second end and a longitudinally expandable section. The first end of the elongate ribbon may be connected to the longitudinally expandable section. The elongate ribbon may include a barb formed therefrom. The stent may include at least one aperture for receiving a release wire therethrough, and wherein the stent defines an outer perimeter, the anchor being restrained within the outer perimeter by the release wire in the delivery configuration, the anchor extending outward radially from the outer perimeter in the expanded configuration.

In another aspect, the present disclosure provides a medical device system which includes a medical device and a release wire. The medical device may be movable between a delivery configuration and an expanded configuration and may include an anchor comprising an elongate ribbon extending from a first end to a second end and a longitudinally expandable section defining at least one curve. The first end of the elongate ribbon may be connected to the longitudinally expandable section. The elongate ribbon may include a barb formed therefrom. The medical device may include at least one aperture for receiving the release wire therethrough. The medical device defines an outer perimeter, the anchor being restrained within the outer perimeter by the release wire in the delivery configuration, and the anchor extending outward radially from the outer perimeter in the expanded configuration.

Further objects, features and advantages of this system will become readily apparent to persons skilled in the art after a review of the following description, with reference to the drawings and claims that are appended to and form a part of this specification.

### BRIEF DESCRIPTION OF THE DRAWINGS

FIG. 1 is a perspective view of an anchor for a medical device being formed from a portion of said medical device, and being constructed in accordance with the principles of the present disclosure;

FIG. 2 is a perspective view of the completed anchor of FIG. 1, including an optional radiopaque marker;

FIG. 3 is a perspective view of an anchor being shaped by a mandrel in accordance with the principles of the present disclosure;

FIG. 4A is a perspective view of an anchor constructed in accordance with the principles of the present disclosure in a remembered state or collapsed state;

FIG. 4B is a perspective view of the anchor of FIG. 4A in an expanded state;

FIG. 5 is a side view of a medical device illustrating a potential location for the anchors according to the present disclosure;

FIGS. 6A-6D are side views illustrating several steps in the deployment of an anchor constructed in accordance with the principles of the present disclosure;

FIGS. 7A-7E are side views illustrating several steps in the repositioning or retrieval of a medical device which includes an anchor constructed in accordance with the principles of the present disclosure;

FIGS. 8-10 are embodiments of devices that include anchors constructed in accordance with the principles of the present disclosure;

FIGS. 11A-11D illustrate steps of introducing and positioning a medical device having anchors as described in the present disclosure in the lumen of a body vessel to be treated;

FIGS. 12A-12D illustrate steps of expanding a medical device having anchors as described in the present disclosure within a body lumen and anchoring said device to the walls of the body lumen;

FIGS. 13A-13D illustrate steps of deploying or implanting a medical device having anchors as described in the present disclosure within a body lumen;

FIG. 14 is a cutaway view illustrating a delivery assembly for delivering a medical device constructed in accordance with the principles of the present disclosure;

FIG. 15 is a perspective view of an anchor for a medical device formed from a portion of said medical device in accordance with the principles of the present disclosure;

FIG. 16 is a perspective view of an anchor being shaped by a mandrel in accordance with the principles of the present disclosure;

FIG. 17A is a perspective view of an anchor constructed in accordance with the principles of the present disclosure in a remembered state or expanded state;

FIG. 17B is a perspective view of the anchor of FIG. 17A in a restrained or delivery state with a release wire;

FIG. 17C is a perspective view of the anchor of FIG. 17B reverting to the expanded state upon withdrawal of the release wire;

FIG. 17D is a perspective view of the anchor of FIG. 17C with the release wire entirely removed;

FIG. 18 is a perspective view of another anchor constructed according to the present disclosure;

FIG. 19 is a view of a stent graft interacting with a device having anchors in accordance with the principles of the present disclosure;

FIGS. 20A-20D are side views illustrating several steps in the deployment of an anchor constructed in accordance with the principles of the present disclosure;

FIGS. 21A-21D are side views illustrating several steps in the deployment of an anchor constructed in accordance with the principles of another embodiment of the present disclosure;

FIGS. 22A-22E are embodiments of devices that include anchors constructed in accordance with the principles of the present disclosure;

FIG. 23 is a schematic of a delivery system in an aorta illustrating several features of delivery of a device constructed in accordance with the principles of the present disclosure;

FIGS. 24A-24D illustrate steps of introducing and positioning a medical device having anchors as described in the present disclosure in the lumen of a body vessel to be treated;

FIGS. 25A-25D illustrate steps of expanding a medical device having anchors as described in the present disclosure within a body lumen and anchoring said device to the walls of the body lumen;

FIGS. 26A-26D illustrate steps of deploying or implanting a medical device having anchors as described in the present disclosure within a body lumen;

FIG. 27 is a cutaway view illustrating a delivery assembly for delivering a medical device constructed in accordance with the principles of the present disclosure;

FIG. 28A is a cutaway view illustrating a delivery system for delivering a medical device constructed in accordance with another embodiment of the present disclosure;

FIGS. 28B and 28C are a close-up views of portions of the delivery system depicted in FIG. 28A;

FIGS. 29A-29H illustrate steps in a method of delivering a medical device using a delivery assembly according to the principles of the present disclosure;

FIG. 30A-30L illustrate steps in another method of delivering a medical device using a delivery assembly according to the principles of the present disclosure;

FIGS. 31A and 31B are close-up views of wires of a medical device system;

FIGS. 32A-32D illustrate views of a delivery system in which a retention wire is employed;

FIGS. 33A-33D illustrate views of another embodiment of a delivery system in which a retention wire is employed;

FIG. 34A is a perspective view of a delivery assembly having shared wire lumens for retention and chocking wires constructed in accordance with the principles of another embodiment of the present disclosure;

FIG. 34B is a cross-sectional view of the delivery assembly of FIG. 34A taken at line 34B;

FIG. 35A is a perspective view of a delivery assembly having separate wire lumens for retention and chocking wires constructed in accordance with the principles of another embodiment of the present disclosure;

FIG. 35B is a cross-sectional view of the delivery assembly of FIG. 35A taken at line 35B;

FIGS. 36A-36C illustrate steps of expanding a medical device having anchors by a delivery system as described in the present disclosure;

FIGS. 37A-37C illustrate steps of expanding a medical device having anchors by a delivery system as described in the present disclosure; and

FIGS. 38A-38F illustrate steps of expanding and releasing a medical device having anchors in a delivery system as described in the present disclosure.

### DETAILED DESCRIPTION

The drawings are purely schematic illustrations of various aspects of the invention and are not necessarily to scale, unless expressly stated.

The terms “substantially” or “about” used herein with reference to a quantity includes variations in the recited quantity that are equivalent to the quantity recited, such as an amount that is equivalent to the quantity recited for an intended purpose or function. “Substantially” or derivatives thereof will be understood to mean significantly or in large part.

As used herein, the term “delivery assembly” refers to a device that is introduced from outside of the body of a patient and into a body lumen to deliver an implant to said body lumen. The term “delivery system” refers to the delivery assembly and the implant as a unit. For example, an implant in its delivery configuration, placed over the delivery assembly, together with the delivery assembly defines the delivery system.

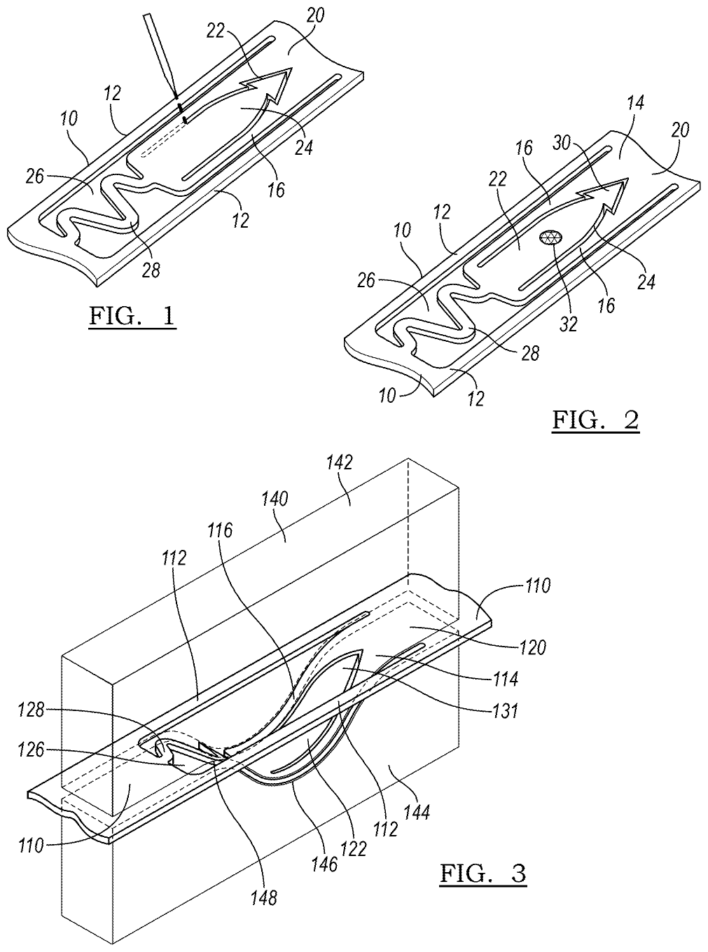

FIG. 1 illustrates a step in the manufacture of a medical device 10 having an anchor 20 formed in accordance with the principles of the present disclosure. The anchor 20 may be cut, such as by laser cutting, from a portion of the medical device 10 to define the various portions of the anchor 20. In one embodiment, the medical device 10 may be a stent, and the portion from which the anchor 20 is formed may be a strut 12.

In one embodiment, the medical device 10 may be a self-expanding stent. Such a stent may be formed of a shape memory material, including a biocompatible shape memory alloy. One such alloy may be a nickel-titanium alloy that allows a device to be manufactured to recover a previous shape. The recovery of this shape may be driven by an increase in temperature, such as at body temperature (referred to as the shape memory effect) or by the removal of an applied stress (the superelastic effect).

FIG. 2 illustrates the complete anchor 20 as manufactured in FIG. 1. The anchor 20 may include a ribbon 16 and a longitudinally expandable, or curved, portion 28. The ribbon 16 may be an elongated ribbon which is bounded by gaps 17. The ribbon 16 has a first end 18 and extends to a second end 30, which is defined by an imaginary line that runs between the ends of gaps 17 along the surface of the device 10. The ribbon 16 includes barb 22, which may be formed or cut from within the ribbon 16 by cut 24.

The longitudinally expandable portion 28 is attached to the ribbon 16 at first end 18 and extends, in the illustrated embodiment, in a curved or sinuous fashion to third end 21. The longitudinally expandable portion may define a plurality of curves or peaks as it proceeds from the third end 21 to the first end 18. In the illustrated embodiment, three curves are defined, but in alternative designs, more or fewer curves may be employed. The longitudinally expandable portion is bounded by spaces 26 which are contiguous with gaps 17. Overall, the anchor extends from third end 21 of the longitudinally expandable portion 28 to second end 30.

Optionally, the anchor 20 may include a radiopaque marker 32 so that the state of the anchor can be monitored during deployment and/or retraction. The radiopaque marker 32 may comprise gold, or tungsten, or any other radiopaque material as is known in the art. The radiopaque marker 32 may be placed on the body of barb 22 or any other suitable location on the anchor 20.

Optionally, the anchor 20 may include a hook 25 at the second end 23 of the ribbon 16 for better anchorage in the vessel wall. As illustrated in FIG. 2, this hook 25 may take on the shape of an arrowhead, and may be flexible further radially outward from the remainder of the body of anchor 20 in an expanded or deployed configuration. However, as the hook 25 represents an extra step in manufacturing, it may not be selected for all applications.

FIG. 3 illustrates another embodiment of an anchor 120 of a device 110. Device 110 has and anchor 120 that lacks a hook as described above. FIG. 3 illustrates a step of shaping the anchor 120 of the self-expanding device 110 by using a two-part mandrel 140. The mandrel 140 has a top half 142 having a convex curved portion 148, and a bottom half 144 having a concave curved portion 146. When the anchor 120 is placed between the concave curved portion 146 and the convex curved portion 148, the anchor 120 becomes disposed within an interior portion of the device 110. For instance, if the device 110 is a stent, the anchor 120 will bow inward within the lumen of the stent, or within the outer perimeter defined by the tubular body of the stent 110. The device 110 may then be heated to a temperature effective to define a remembered state of the anchor 120, wherein it is disposed within the interior of the device 110.

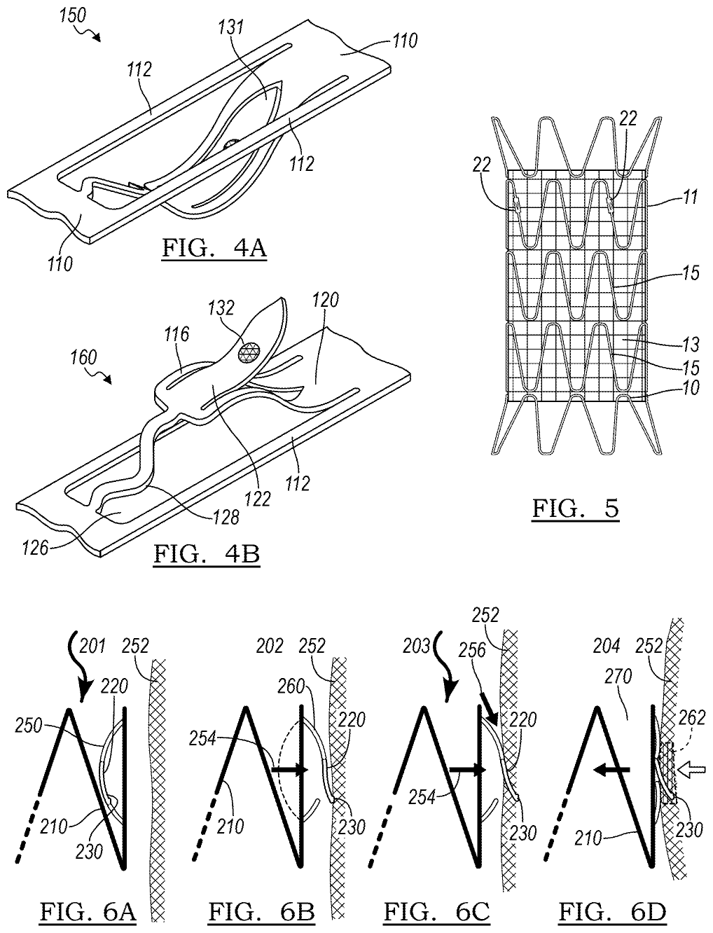

FIG. 4A illustrates the remembered state 150 of the anchor 120 as formed in FIG. 3. This configuration represents a low energy or relaxed state, to which the anchor 120 tends to return when not under the influence of an external force. Remembered state 150 also represents a delivery state of the anchor 120 wherein anchor 120 is retracted within the lumen of the device 110. However, in this embodiment, anchor 120 will remain inside the perimeter, or the lumen, even of the device 110 in the expanded state, and even if no external force is applied to eject the anchor 120 beyond the nominal outer surface of the device 110.

FIG. 4B illustrates the anchor 120 of FIG. 4A in the expanded state 160. The anchor 120 curves out radially, beyond the circumferential perimeter defined by the remainder of strut 112. The curve of the longitudinally expandable portion 128 allows the anchor to deform to the expanded state 160 by straightening as shown in FIG. 4B, and to interconvert between the two previously-noted specific shapes: the low energy, relaxed delivery state 150, and the higher-energy, expanded, deployed state 160. Despite a preference for the delivery state, the anchor 120 is bistable; although the remembered state 150 is a lower-energy state, the device 110 in the deployed state 160 is stable when the anchor is embedded in a wall surrounding a body lumen.

In FIG. 4B, the barb 122 is flexed radially outward. This figure represents a state in which the anchor 120 has been pushed out by an additional, externally provided radial force; for example, by the expansion of a balloon of a delivery assembly. In some embodiments, the tip of the barb 122 may represent the most radially-outwardly disposed portion of the expanded device.

FIG. 5 illustrates a stent graft 11. A self-expanding stent 10 including at least one anchor 20 as described in accordance with the principles of the present disclosure may interact with such a stent graft 11 in a number of ways. In a first embodiment, the stent 10 may be a bare metal stent which may be implanted within a body lumen to be treated prior to delivery of a standard endograft, the bare metal stent paving the way and preparing the geometry of the vessel as will be described below. In such an embodiment, the stent will be generally located more proximate the proximal or the distal end of the stent graft, which will be fit within the lumen of the paving stent. In another embodiment, the stent 10 will be incorporated into the stent graft 11 itself, by stitching the stent 10 to the graft material 13. The stent graft 11 additionally includes further stent rings 15 as part of its body.

FIGS. 6A-6D illustrate steps in the deployment of a device 210 having at least one anchor 220 as constructed in accordance with the principles of the present disclosure. As shown in FIG. 6A, in step 201, the device 210 is introduced into a lumen of a vessel 252. The vessel 252 in this embodiment is a blood vessel, with blood flow in the direction B, as illustrated by the arrow. The anchor 220 is in its delivery, or collapsed, configuration 250.

In FIG. 6B, step 202 illustrates the result of the application of a radial expansion force 254 (such as by an expandable balloon, not illustrated for clarity) to the point that the anchor 220 adopts its expanded configuration 260. In this step, the point 230 of the barb of the anchor 220 comes in contact with the wall of vessel 252. In FIG. 6C, radial expansion force 254 continues to be applied, causing the anchor 220 to be driven into the wall of the vessel 252 (see piercing force 256). In this step, the anchor 220 may attempt to return to its remembered state, thereby pulling the vessel wall inward and more securely attaching the device within the lumen. Finally, in step 204 of FIG. 6D, the radial deployment force is removed, as the device 210 attains its deployed state 270.

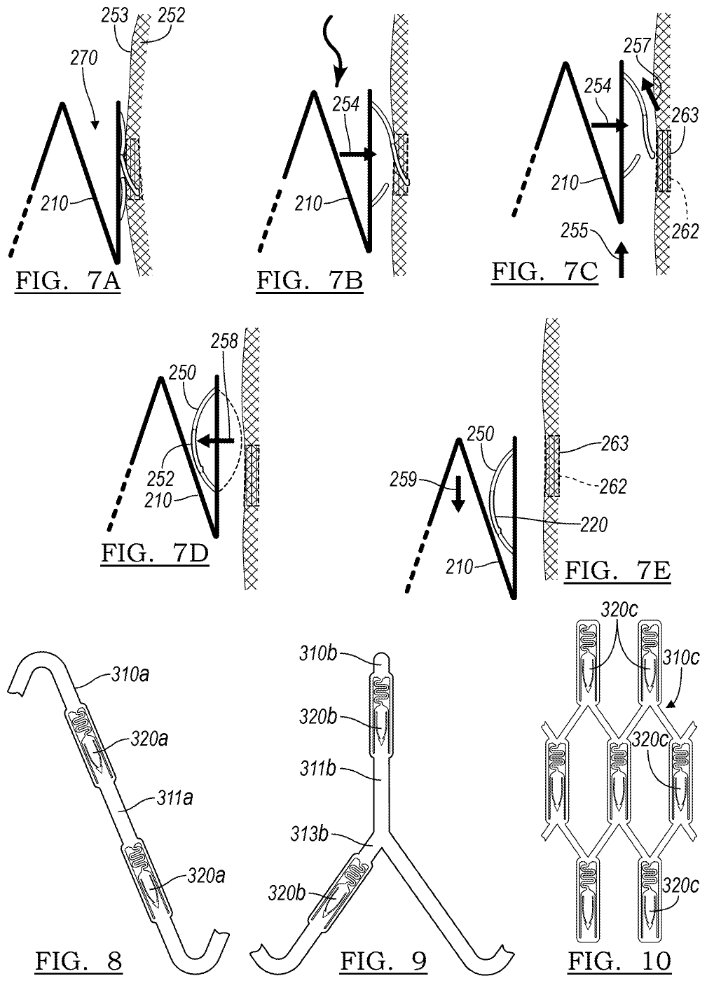

FIGS. 7A-7E illustrate procedural steps in repositioning or withdrawing the device 210. For convenience, the embodiment and schematic illustrations as in FIGS. 6A-6D are adopted for FIGS. 7A-7E.

FIG. 7A illustrates the deployed state 270 of device 210. The anchor 220 is firmly in place, implanted in the wall 253 of vessel 252.

In FIG. 7B, an outward radial deployment force 254 is once again applied, expanding the device 210 and moving it radially outward relative to the deployed anchor 220. This effectively amounts to a redeployment, and allows the tissue to be released from the anchor 220. The repositioning procedure continues in FIG. 7C, where a force 255 in the direction of one of the ends of the device 210 (an upward force as illustrated) is employed, releasing the tissue at the previous implantation site 263 from the anchor 220.

In FIG. 7D, the removal of the radial deployment force is represented by arrow 258. With this external force removed, the anchor 220 readopts its collapsed, or delivery configuration 250, the low-energy state. Finally, in FIG. 7E, the user is able to reposition the device 210 by applying a repositioning force 259. This step may then be followed by the steps of FIGS. 6A-6D, wherein a radial deployment force is exerted on the device 210, or the device may instead be withdrawn from the body. The device 210 may be repositioned as many times as is necessary.

The anchors as described herein may be employed on a variety of medical devices to be implanted within a body lumen. In the case where the device is a cannula-cut, self-expanding stent, the anchors may be cut from struts of the stent. In some embodiments, a single anchor may be cut from a particular strut.

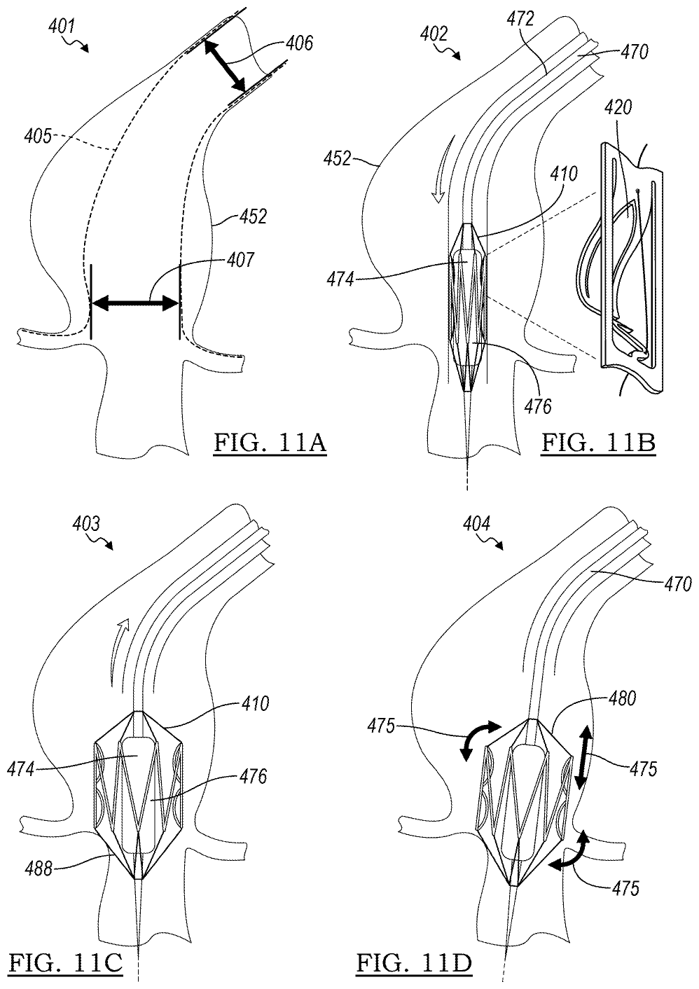

FIGS. 8-10 illustrate different embodiments for providing anchors on a device. In FIG. 8, two anchors 320a are provided in a single strut 311a. In FIG. 9, an anchor 320b is provided in a strut 313b of a stent ring, and another anchor 320b is provided in an interconnecting strut 311b of stent 310b. In FIG. 10, a plurality of anchors 320c are provided in a variety of struts of device 310c. FIGS. 8-10, like the other drawings presented herewith, are not necessarily to scale.

In another embodiment, and as shown in FIGS. 11-13, a paving stent 410 may be deployed to a body vessel to be treated.

FIGS. 11A-11D illustrate steps of introducing an implantable medical device to a body vessel to be treated. One such body vessel can be the aorta, at the site of an aortic aneurysm.

In FIG. 11A, in step 501, a desirable landing zone 405 for an endoluminal graft is illustrated in vessel 452. To prepare the vessel 452 for the implantation of a graft, a distal landing zone 406 and a proximal landing zone 407 are identified. In accordance with the principles of the present disclosure, at least one of these zones, or both, will be treated by implantation of a paving stent 410.

In FIG. 11B, the paving stent 410 is delivered in its compressed state within outer sheath 471 of delivery assembly 470. The stent 410 is disposed over and around inflatable balloon 474 of balloon catheter 472, with the balloon 472 in its deflated state 476. As shown in the inset, the anchor 420 is in its delivery configuration 450, within the lumen of stent 410. The stent 410 is retained by control wires 480.

In FIG. 11C, in step 503, the outer sheath 471 is pulled back by the operator to allow the stent 410 to adopt its expanded configuration 488. In FIG. 11D, in step 504, the operator optionally uses a visualization technique to ascertain the position of the device as it is being deployed, making positional adjustments 475 as needed.

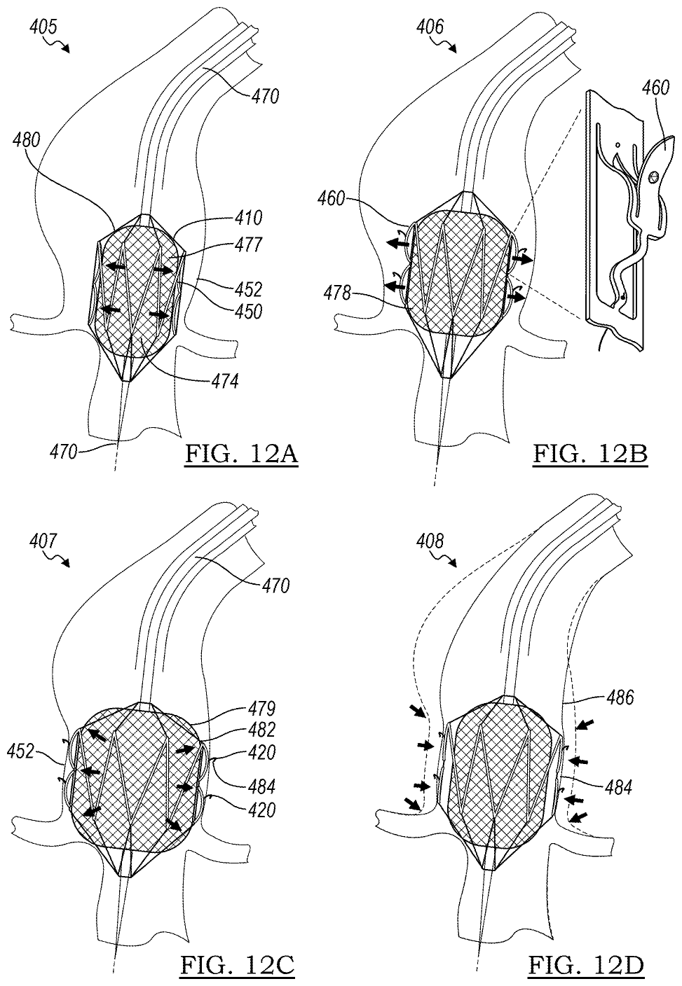

FIGS. 12A-12D represent further steps of expanding the device. In FIG. 12A, the balloon 474 begins to be inflated to adopt partially inflated condition 477, and in FIG. 12B, in step 506, the balloon 474 is expanded into further inflated condition 478. In this condition, the stent 410 has been sufficiently expanded to allow the anchors 420 to achieve their expanded state 460, and rather than being inside the lumen of the stent 410, the anchors now extend radially outward from the stent body. In some embodiments, the anchors 420 may, in expanded state 460, represent the outermost portion of the stent 410.

In step 507, shown in FIG. 12C, expansion continues, and the balloon 474 reaches its fully inflated condition 479. The anchors 420 have now engaged the wall of vessel 452, and stent 410 is now in its deployed state 484.

A final expansion step 508 is shown in FIG. 12D. In some cases, as the balloon 474 is deflated to a partially inflated condition 484, the stent 410 may also contract in the radial dimension from its overexpanded condition and, by virtue of the anchors 420 having been embedded in the wall of vessel 452, may pull in the vessel 452 with it, reshaping the vessel such that it takes on remodeled state 486. A reshaped vessel of about 20 millimeters (mm) to about 30 mm in length may provide a more symmetrical cross-section than the vessel in its initial, diseased state. This symmetrical, remodeled vessel portion can serve as a stable platform for the secondary deployment of a conventional endovascular stent in order to provide for reliable treatment of challenging aneurysm and dissection cases while reducing the risk of future endoleak, migration, and propagation of the aneurysm sac. With the ability to remodel an eccentrically-shaped sac to a more symmetric tubular landing zone, the stent 410 may also be used in conjunction with a preparatory stent for a transcatheter heart valve deployment, or similar diseases wherein mechanical retraction of tissue is desirable.

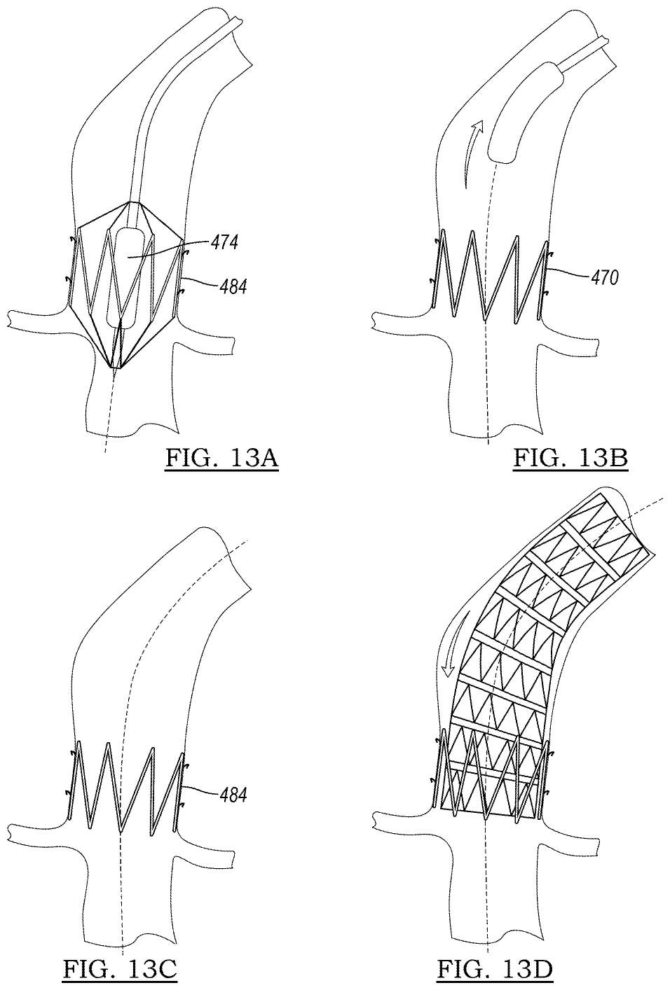

FIGS. 13A-13D illustrate steps of withdrawing the delivery system 470 from the body vessel 452. In step 509, shown in FIG. 13A, the balloon 474 is deflated, the stent 410 remaining in its deployed state 484 about the balloon 474.

In step 510, shown in FIG. 13B, the release wires 480 have been disengaged from the stent, and the delivery assembly is withdrawn, optionally over a wire guide. The delivery assembly 470 has been completely removed in step 511, in FIG. 13C. Finally, with the proximal landing zone have been paved by paving stent 410, the final step of delivery includes the introduction of a secondary device, endograft 411.

In some embodiments, the distal landing zone 407 would have been paved by a stent device instead of, or in addition to, the proximal landing zone 406, as was illustrated in FIGS. 11-13.

The above is an example of how the dynamic anchor, as opposed to one that is static, allows the user the choice of when to deploy the anchors in situ to affect landing and fixation. As a result, the user is able to adjust the location of the landing zone for the implant while it is expanded, but still not released from the delivery device, thereby minimizing the risk that the vessel will be damaged by anchors as it is manipulated. Furthermore, the dynamic nature of the anchors allows for de-anchoring of the device so that it may be repositioned in the lumen, or removed from the patient altogether.

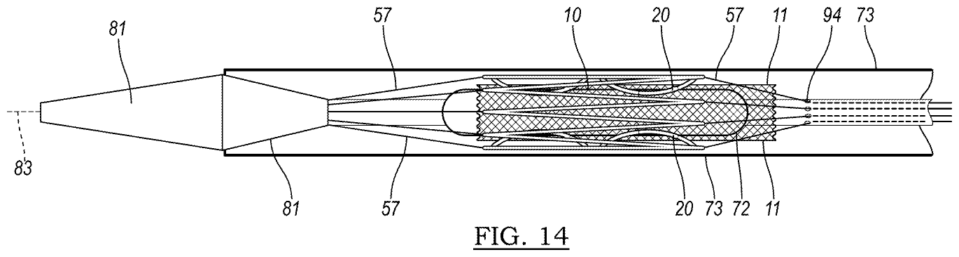

FIG. 14 illustrates one embodiment of a delivery system 70 which may be used to deliver a device 10 as described herein. The device 10, with anchors 20, is illustrated in FIG. 14 as being positioned over balloon 74 of balloon catheter 72. Optionally, if the device 10 is a stent that has been incorporated into a stent graft, stent graft 11 is visible in the drawing. The stent may be restrained by control wires 80, which are fed through apertures 94 and are controllable, individually or all together, by the user. The delivery system 70 optionally includes a tip 81 for guiding the assembly through the vasculature, and may be delivered over wire guide 83.

Another aspect of the disclosure is illustrated in FIG. 15. FIG. 15 illustrates a portion of a medical device 510 having an anchor 520 formed in accordance with the principles of the present disclosure. The anchor 520 may be formed by cutting, such as by laser cutting, from a portion of the medical device 510 to define the various portions of the anchor 520. In one embodiment, the medical device 510 may be a stent, and the portion from which the anchor 520 is formed may be a strut 512.

In one embodiment, the medical device 510 may be a self-expanding stent. Such a stent may be formed of a shape memory material, including a biocompatible shape memory alloy. One such alloy may be a nickel-titanium alloy that allows a device to be manufactured to recover a previous shape. The recovery of this shape may be driven by an increase in temperature, such as at body temperature (referred to as the shape memory effect) or by the removal of an applied stress (the superelastic effect).

The anchor 520 may include a ribbon 516 and a longitudinally expandable, or curved, portion 528. The ribbon 516 may be an elongated ribbon which is bounded by gaps 517. The ribbon 516 has a first end 518 and extends to a second end 523, which is defined by an imaginary line that runs between the ends of gaps 517 along the surface of the device 510. The ribbon 516 includes barb 522, which may be formed or cut from within the ribbon 516 by cut 524.

The longitudinally expandable portion 528 is attached to the ribbon 516 at first end 518 and extends, in the illustrated embodiment, in a curved or sinuous fashion to third end 521. The longitudinally expandable portion may define a plurality of peaks as it proceeds from the third end 521 to the first end 518. In the illustrated embodiment, three curves are defined, but more or fewer may be employed. The longitudinally expandable portion is bounded by spaces 526 which are contiguous with gaps 517. Overall, the anchor extends from third end 521 of the longitudinally expandable portion 528.

The anchor 520 also may include a first aperture 527 and a second aperture 529 defined therethrough. In some embodiments, the first aperture 527 and the second aperture 529 may provide fluid connection between the interior lumen of the device and the environment external to the device 510. In some embodiments, the first aperture 527 may be a proximal aperture, and second aperture 529 may be a distal aperture.

As will be described in detail later, the first and second apertures 527/529 allow a release wire to be threaded through the anchor 520. The terms “release wire” and “control wire” refer to wires that are part of a delivery assembly and allow for manipulation of a medical device within the vasculature of a patient prior to and during deployment, and are used interchangeably in this disclosure.

Optionally, the anchor 520 may include a radiopaque marker 532 so that the state of the anchor can be monitored during deployment and/or retraction. The radiopaque marker 532 may comprise gold, or tungsten, or any other radiopaque material as is known in the art. The radiopaque marker 532 may be placed on the body of barb 522 or any other suitable location on the anchor 520.

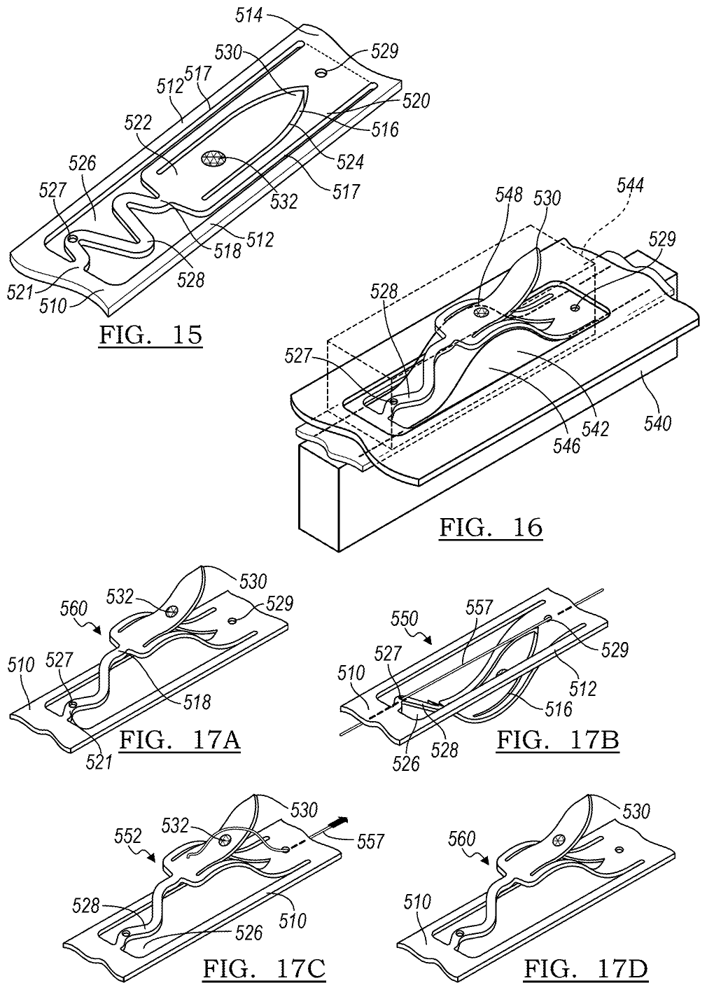

FIG. 16 illustrates a step of shaping an anchor 520 of the self-expanding device 510 by using a two-part mandrel 540. The mandrel 540 has a top half 544 having a concave curved portion 548, and a bottom half 542 having a convex curved portion 546. When the anchor 520 is placed between the concave curved portion 548 and the convex curved portion 546, the anchor 520 becomes disposed radially outward from the remainder of the body of device 510. For instance, if the device 510 is a stent, the anchor 520 will be bent such that it lies outside of the perimeter defined by the tubular body of the stent 510. The device 510 may then be heated to a temperature effective to define a remembered state of the anchor 520, wherein it is disposed outside of the outer perimeter of device 510, and may be restrained with a control wire in the delivery assembly.

FIG. 17A illustrates the remembered state 560 of the anchor 520 as formed in FIG. 16. This configuration represents a low energy or relaxed state, to which the anchor 520 tends to return when not under the influence of an external force, such as from a control wire. Remembered state 550 represents a configuration to which the anchor 520 will return, external to the outer perimeter defined the device 510. In the case of a stent, this outer perimeter is defined by the generally tubular or cylindrical profile of the stent.

FIG. 17B illustrates the anchor 20 of FIG. 17A in the retracted, or delivery state 550. The anchor 520 curves inward radially, within the lumen of device 510 and the circumferential perimeter defined by the remainder of strut 512. The inward curve of anchor 520 is maintained by tension from control wire 557, which is threaded through the first aperture 527 and the second aperture 529 and pulled taut to force the anchor 520 inward.

The curve of the longitudinally expandable portion 528 is straightened in the low energy configuration 560, which allows the anchor 520 to deform to the expanded state 560 by straightening as shown in FIG. 17A, and to interconvert between the two previously-noted specific shapes: the low energy, relaxed deployed state 560, and the higher-energy, restrained state 550.

FIG. 17B illustrates a restrained or delivery state 550 in which the barb 522 is curved radially outward. The control wire 557 runs through first aperture 527 and beyond third end 521, and through second aperture 29 and beyond second end 523. In FIG. 17C, the operator has pulled the control wire 557 distally, releasing it from the first aperture 527, and allowing the anchor 520 to adopt a partially released state 552. FIG. 17D shows the anchor 520 fully released and in expanded configuration 560.

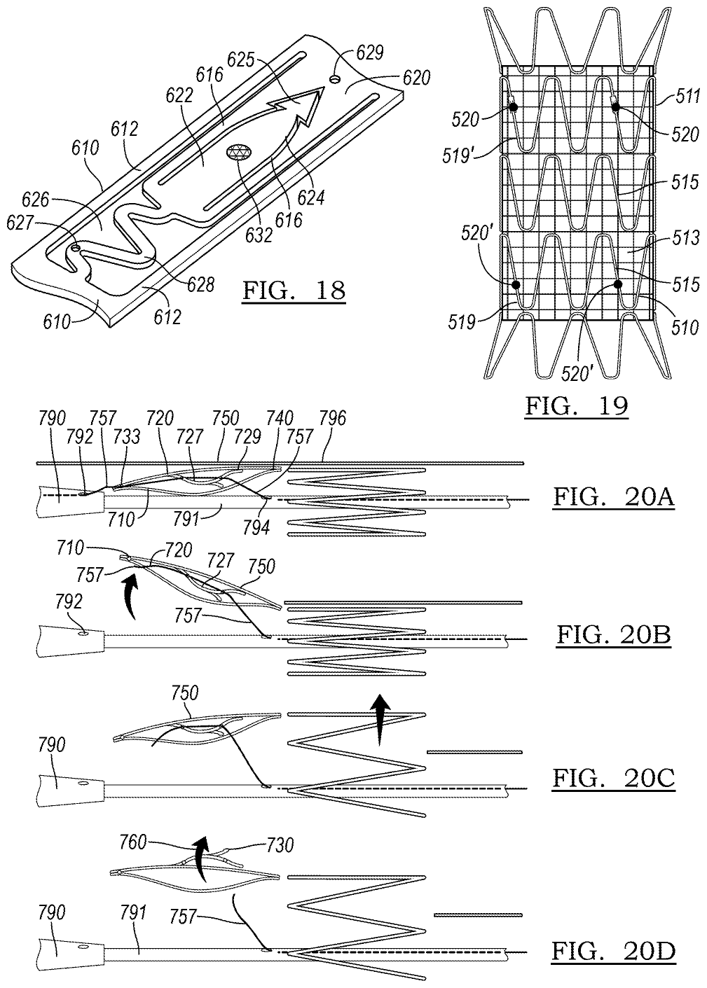

Another embodiment of an anchor 620 is shown in FIG. 18. Optionally, the anchor 620 may include a hook 625 at the end of the ribbon 616 for better anchorage in the vessel wall. As illustrated in FIG. 18, this hook 625 may take on the shape of an arrowhead, and may be flexible further radially outward from the remainder of the body of anchor 620 in an expanded or deployed configuration. However, as the hook 625 represents an extra step in manufacturing, it may not be used in all situations.

FIG. 19 illustrates a stent graft 511. A self-expanding stent 510 including at least one anchor 20 as described in accordance with the principles of the present disclosure may interact with such a stent graft 511 in a number of ways. In a first embodiment, the stent 510 may be a bare metal stent which may be implanted within a body lumen to be treated prior to delivery of a standard endograft, the bare metal stent paving the way and preparing the geometry of the vessel as will be described below. A bare metal stent may be deployed over a region where perfusion of side branch vessels is anticipated. In such an embodiment, the stent will be generally located more proximate the proximal or the distal end of the stent graft, which will be fit within the lumen of the paving stent. In another embodiment, the stent 510 will be incorporated into the stent graft 511 itself, by stitching the stent 510 to the graft material 513. The stent graft 511 additionally includes further stent rings 515 as part of its body.

In some embodiments, the anchor 520 resides on a stent ring 510 that makes up a portion of an endograft, such as stent graft 511 shown in FIG. 19. The stent graft may include a stent ring 510 having anchor 520 as its proximal stent ring 519. In another embodiment, the distal stent ring 519′ may include an anchor 520′ as described herein. In another embodiment, both the proximal stent ring 519 and the distal stent ring 519′ will include anchors 520 and 520′, respectively. In another embodiment, more stent rings, intermediate between the proximal stent ring 519 and the distal stent ring 519′ may include an anchor. The stent rings 510 will be incorporated into the stent graft 511 itself, by stitching the stent 510 to the graft material 513 as is known in the art. The stent graft 511 may additionally include further stent rings 515, not including anchors, as part of its body.

FIGS. 20A-20D illustrate steps in the deployment of a device 710 having at least one anchor 720 as constructed in accordance with the principles of the present disclosure. As shown in FIG. 20A, the device 710 is introduced within a sheath 796. The anchor 720 is in its delivery, or collapsed, configuration 750, restrained by control wire 757.

In the embodiment of FIG. 20A, the control wire 757 controls the release of both the anchor 720 and the overall device 710. The control wire 757 may run through the tip 790 of the delivery assembly, emerging through aperture 792, and being threaded to an aperture 733 at the end of the device 710. The control wire 757 then passes through first aperture 727 of the anchor 720, and through second aperture 729, with sufficient tension to retain the anchor 720 in its delivery state 750. The retention wire 757 then passes through the port 794 of inner catheter 791. The control wire 757 can be manipulated by the user via a handle as is known in the medical device delivery art. An assembly with a single control wire 757 may contribute to a lower-profile device than an embodiment in which multiple wires are employed.

In FIG. 20B, the control wire 757 has been pulled to the right of the figure out of aperture 792 and aperture 733 of device 710, thereby releasing the end of the device 710. The sheath 796 has also been pulled to the right, allowing the device 710 to partially radially expand. The control wire 757 still runs through both apertures 727/729 of the anchor 720, so it remains in delivery state 250.

In FIG. 20C, the sheath 796 has been pulled yet further, allowing more of the device 710 to expand in the radial dimension. Finally, FIG. 20D shows a state in which the control wire 757 has been withdrawn from both apertures 727/729, allowing the anchor 720 to convert to expanded state 760 and engage the vessel wall with tip 730.

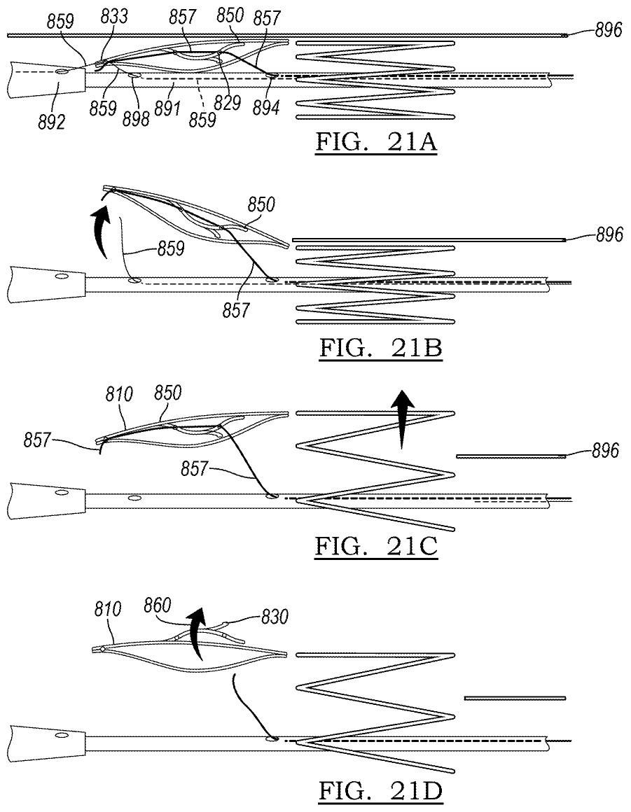

FIGS. 21A-21D illustrate another embodiment of a delivery assembly for delivery of a device 810 having anchors 820. In the embodiment depicted in FIG. 21A, a device control wire 859 is provided as a part distinct from anchor control wire 857. The device control wire 859 emerges from tip aperture 892, passes through aperture 833 on the end of the device 810, and re-enters the inner catheter 890 through port 898 as it extends to the handle, where it may be controlled by an operator. In FIG. 21A, the anchor 820 remains in its delivery configuration 850. Anchor control wire 857 likewise emerges from an aperture, in some embodiments such as aperture 892, and passes through the holes 827 and 829 (not labeled in all figures; analogously positioned as holes 527/529 of FIG. 15) of the anchor 820 before re-entering inner catheter 890 through port 394 as it extends to a handle, where it may be controlled by an operator.

In FIG. 21B, the device control wire 859 has been pulled to the right by the operator, while the anchor control wire 857 remains in place. The outer sheath 896 of the delivery assembly has also been withdrawn in the same direction. As a result of both of these actions, the end of device 810 is no longer tethered to the delivery assembly, but the anchor 820 remains in the delivery configuration 850 and remains within the lumen of device 810.

In FIG. 21C, the operator has withdrawn outer sheath 896 further, causing further radial expansion of the previously-compressed self-expanding device 810. This causes more of the device 810 to expand, and may assist in properly positioning the device 810 in the body lumen. Finally, in FIG. 21D, the device control wire 857 is withdrawn from anchor 820, allowing anchor 820 to adopt its expanded, deployed configuration 860.

The anchors as described herein may be employed on a variety of medical devices to be implanted within a body lumen. In the case where the device is a cannula-cut, self-expanding stent, the anchors may be cut from struts of the stent. In some embodiments, a single anchor may be cut from a particular strut.

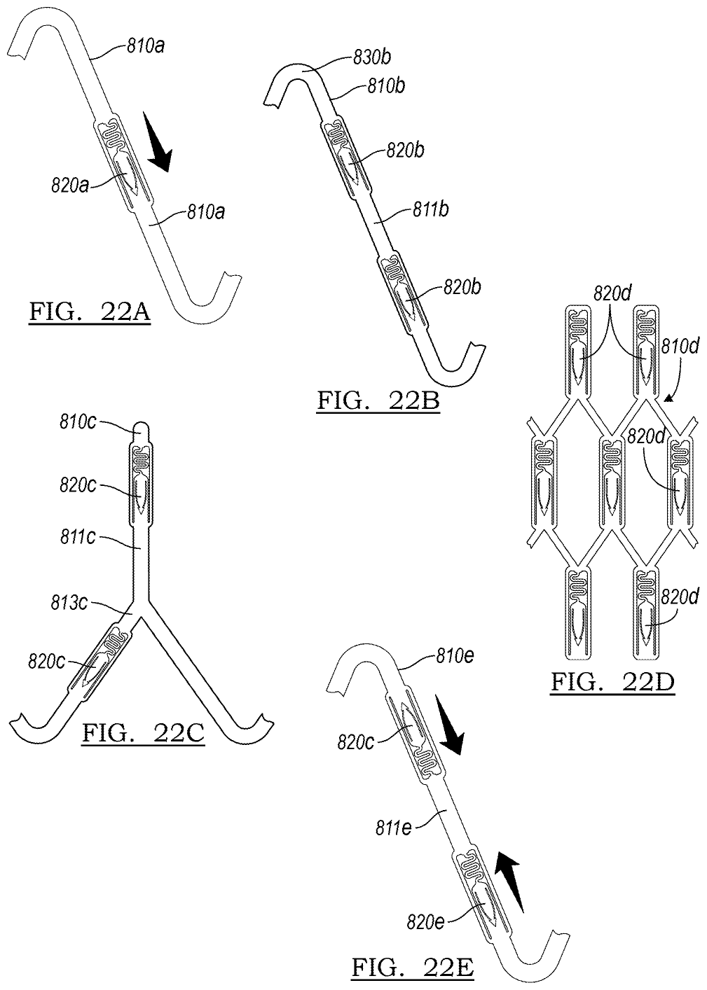

FIGS. 22A-22E illustrate different embodiments for providing anchors on a device. In FIG. 22A, one anchor 820a is provided in a single strut 811a. In FIG. 22B, two anchors 820b are provided in a single strut 811b. In FIG. 22C, an anchor 820c is provided in a strut 813c of a stent ring, and another anchor 820c is provided in an interconnecting strut 811c of stent 810c. In FIG. 22D, a plurality of anchors 820d are provided in a variety of struts of device 810d. In FIG. 22E, anchors 820e are positioned as in FIG. 22B, but with their tips pointed in opposing directions, rather than in the same orientation as in FIG. 22B. FIGS. 22A-22E, like the other drawings presented herewith, are not necessarily to scale.

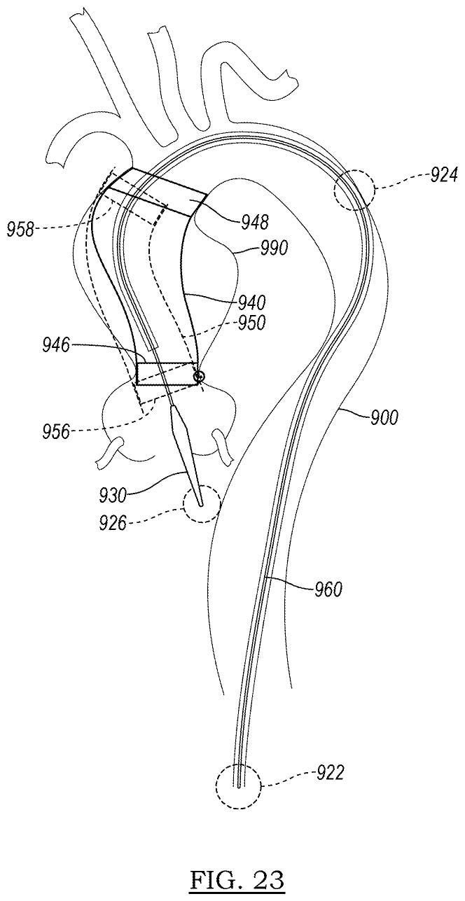

One context in which anchors constructed in accordance with the principles of the present disclosure may be employed is in a stent that paves the way for an endograft to be implanted in the aorta of a patient. FIG. 23 depicts an aorta 900 into which a delivery system 960 has been inserted. The aorta 900 has an aortic aneurysm 990, which is to be treated by the endograft. Due to the complex, arcing geometry of the aorta 900, a graft device may land in a suboptimal orientation upon delivery, which in the long term may lead to type I endoleak or migration of the implant.

One contributing factor in suboptimal landing is lack of control when deploying the stent to achieve a competent seal zone. In addition, the burst of elastic energy released from the stent during deployment, as well as that of the anchor barbs, can decrease the precision in controlling the landing orientation to the ideal seal zone during deployment.

The energy associated with expansion of the stent is not the only force that contributes to the ultimate positioning of the device. A user applies a force on the delivery system at zone 922; the delivery system 960 may contact the wall of the aorta at the curve at zone 924; and the tip 930 of the delivery system 960 may contact intravascular surfaces such as at zone 926. Each of these may act to destabilize the delivery system 960 and introduce imprecision during deployment of the device. Rather than landing a desirable landing zone 940, having proximal landing zone 946 and distal landing zone 948, the device may instead land in projected landing zone 950, having proximal landing zone 956 and distal landing zone 958. Both zone 958 and 956 have the potential to lead to subpar sealing, particularly when compared to respective zones 948 and 946 in a desirable landing zone 940.

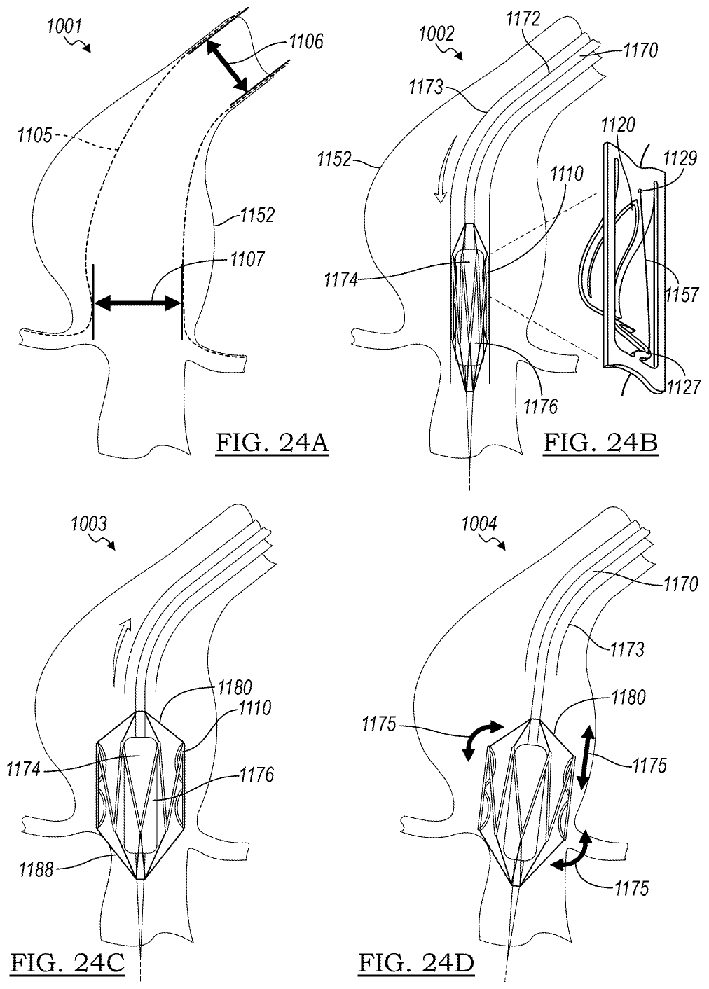

As shown in FIGS. 24-26, a paving stent 1110 may be deployed to a body vessel to be treated to reduce the chance of problems as described in FIG. 23. FIGS. 24A-24D illustrate steps of introducing a device to a body vessel to be treated.

In FIG. 24A, in step 1001, the desired landing zone 1105 for an endoluminal graft is illustrated in vessel 1152. To prepare the vessel 1152 for the implantation of a graft, a distal landing zone 1106 and a proximal landing zone 1107 are identified. In accordance with the principles of the present disclosure, at least one of these zones, or both, will be treated by implantation of a paving stent 1110.

In FIG. 24B, in step 1002, the paving stent 1110 is delivered in its compressed state within outer sheath 1171 of delivery assembly 1170. The stent 1110 is disposed over and around inflatable balloon 1174 of balloon catheter 1172, with the balloon 1172 in its deflated state 1176. As shown in the inset, the anchor 1120 is in its delivery configuration 1150, within the lumen of stent 1110 and restrained by control wire 1157 as it passes through apertures 1127 and 1129. The stent 1110 is retained by control wires 1180.

In FIG. 24C, in step 1003, the outer sheath 1171 is pulled back by the operator to allow the stent 1110 to adopt its expanded configuration 1188. In FIG. 24D, in step 1004, the operator optionally uses a visualization technique to ascertain the position of the device as it is being deployed, making positional adjustments 1175 as needed.

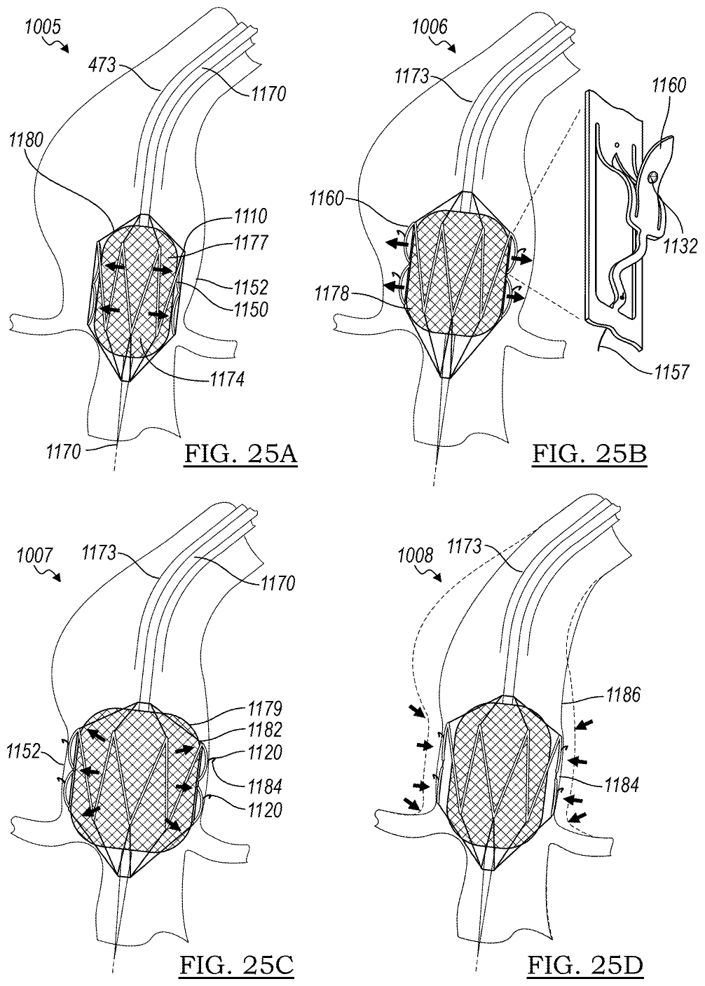

FIGS. 25A-25D represent further steps of expanding the device. In FIG. 25A, in step 1005, the balloon 1174 begins to be inflated to adopt partially inflated condition 1177, and in FIG. 25B, in step 1006, the balloon 1174 is expanded into further inflated condition 1178. In this condition, the stent 1110 has been sufficiently expanded to allow the anchors 1120 to achieve their expanded state 1160, and rather than being inside the lumen of the stent 1110, the anchors now extend radially outward from the stent body. In some embodiments, the anchors 1120 may, in expanded state 1160, represent the outermost portion of the stent 1110.

In step 1007, shown in FIG. 25C, the balloon 1174 reaches its fully inflated condition 1179, overexpanding the stent 1110. The control wire 1157 is withdrawn in this step, allowing the anchors 1120 to engage the wall of vessel 1152, and stent 1110 is now in its deployed state 1184.

A final expansion step 1008 is shown in FIG. 25D. In some cases, as the balloon 1174 is deflated to a partially inflated condition 1184, the stent 1110 may also contract in the radial dimension and, by virtue of the anchors 1120 having been embedded in the wall of vessel 1152, may pull in the vessel with it, reshaping the vessel such that it takes on remodeled state 1186. A reshaped vessel of about 20 millimeters (mm) to about 30 mm in length may provide a more symmetrical cross-section than the vessel in its initial, diseased state. This symmetrical, remodeled vessel portion can serve as a stable platform for the secondary deployment of a conventional endovascular stent in order to provide for reliable treatment of challenging aneurysm and dissection cases while minimizing the risk of future endoleak, migration, and propagation of the aneurysm sac. With the ability to remodel an eccentrically-shaped sac to a more symmetric tubular landing zone, the stent 410 may also be used in conjunction with a preparatory stent for a transcatheter heart valve deployment, or similar diseases wherein mechanical retraction of tissue is desirable.

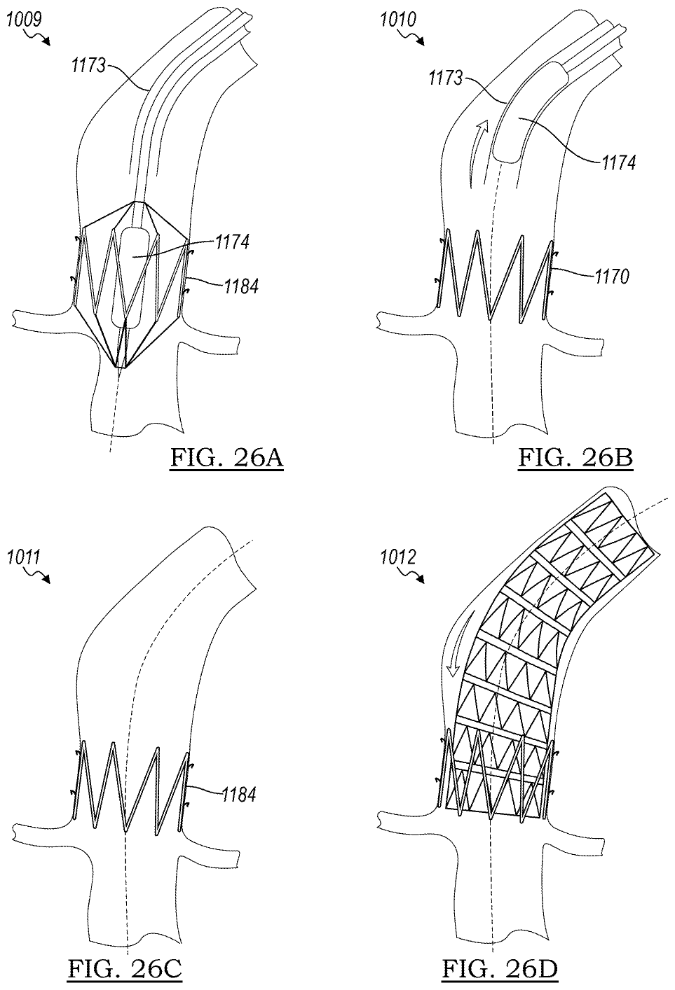

FIGS. 26A-26D illustrate steps of withdrawing the delivery system 1170 from the body vessel 1152. In step 1009, shown in FIG. 26A, the balloon 1174 is deflated, the stent 410 remaining in its deployed state 1184 about the balloon 1174.

In step 1010, shown in FIG. 26B, the release wires 1180 have been disengaged from the stent, and the delivery assembly is withdrawn, optionally over a wire guide. The delivery assembly 1170 has been completely removed in step 1011, in FIG. 26C. Finally, with the proximal landing zone have been paved by paving stent 1110, the final step of delivery as shown in FIG. 26D includes the introduction of a secondary device, endograft 1111, which can occur immediately after withdrawal of the deployment system for the paving stent 1110, or after a period during which vessel remodeling is known to have occurred. In the latter case, it is known that reduction in vessel stress can induce aneurysm sac reduction, which may in turn eliminate the channels created by infolded vessels by the paving stent, thereby minimizing risk of endoleak between the vessel wall to the secondary device.

In some embodiments, the distal landing zone 1107 would have been paved by a stent device instead of, or in addition to, the proximal landing zone 1106, as was illustrated in FIGS. 24-26.

The above is an example of how the dynamic anchor, as opposed to one that is static, allows the user the choice of when to deploy the anchors in situ to affect landing and fixation. As a result, the user is able to adjust the location of the landing zone for the implant while it is expanded, but still not released from the delivery device, thereby minimizing the risk that the vessel will be damaged by anchors as it is manipulated, and maximizing the chance that the device will land in the intended zone. Furthermore, the dynamic nature of the anchors allows for de-anchoring of the device so that it may be repositioned in the lumen, or removed from the patient altogether.

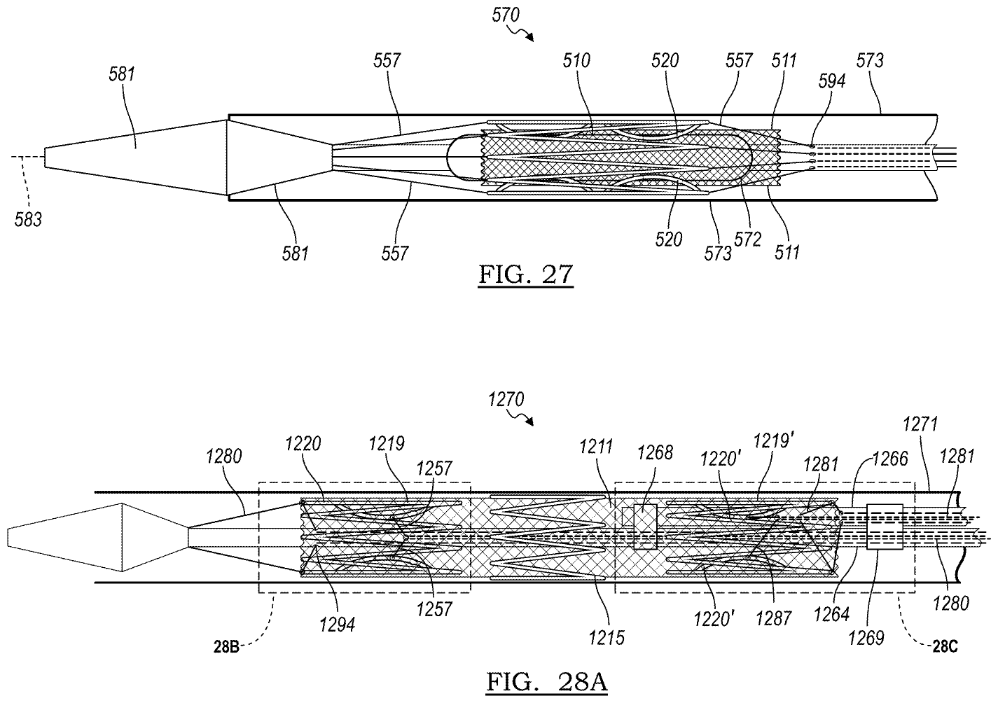

FIG. 27 illustrates one embodiment of a delivery system 570 which may be used to deliver a device 510 as described herein. The device 510, with anchors 520, is illustrated in FIG. 27 as being positioned over balloon 574 of balloon catheter 572. Optionally, if the device 510 is a stent that has been incorporated into a stent graft, stent graft 511 is visible in the drawing. The stent may be restrained by control wires 580, which are fed through apertures 594 and are controllable, individually or all together, by the user. The delivery system 570 optionally includes a tip 581 for guiding the assembly through the vasculature, and may be delivered over wire guide 583.

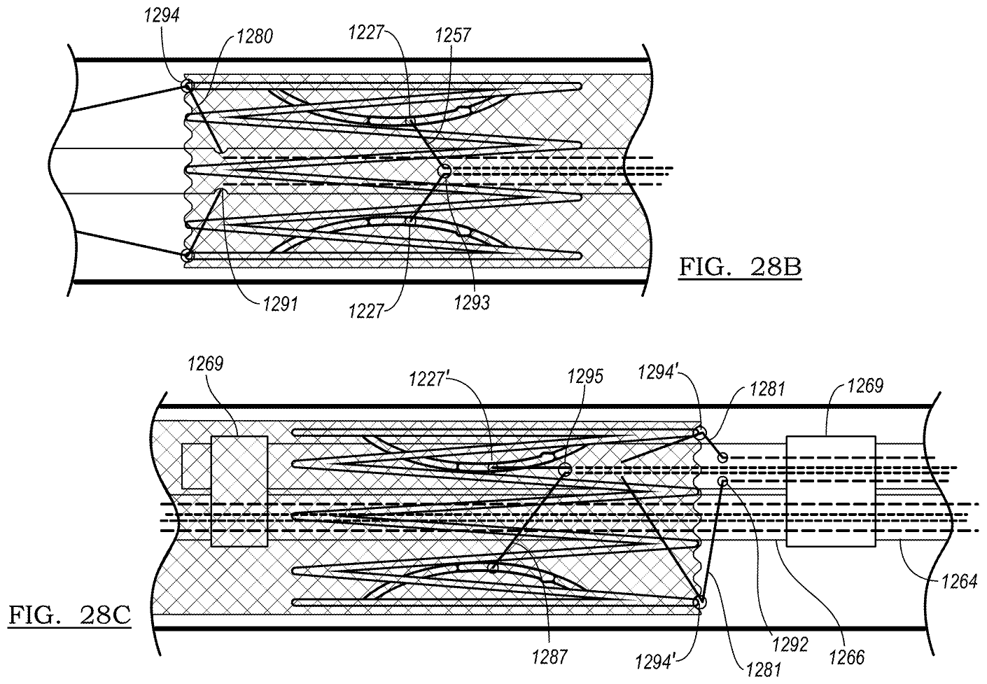

FIG. 28 illustrates another aspect of the present disclosure. FIG. 28A shows a delivery system 1270 which includes stent graft 1211, in which the proximal stent ring 1219 and the distal stent ring 1219′ have controllable anchors 1220 and 1220′ formed therefrom, respectively. FIGS. 28B and 28C are closeup views of the indicated portions of delivery system 1270.

In FIG. 28A, the delivery system 1270 is provided with two inner tubular members. First pusher 1264 is primarily responsible for the placement and control of proximal stent ring 1210 and associated anchor control wires 1257 and stent release wires 1280, whereas second pusher 1266 is primarily responsible for control of distal stent ring 1219′ and associated control wires 1281. The pushers 1264/1266 may optionally be connected via band 1268 and/or band 1269, and the second pusher 1266 may be slidable relative to first pusher 1264. The distal stent control wires 1281, may optionally be provided separately from distal anchor release wires 1287, or may control both anchor and stent release. Packing studies have demonstrated that even with a stent graft in a tightly packed configuration, over 30% of the space of a delivery system remain empty, which suggests that additional delivery elements such as a second pusher could be accommodated in such a system.

As shown in FIGS. 28B and 28C, the stent control wires 1280 may be fed through aperture 1294 of proximal stent 1219, and stent control wires 1281 may be fed through aperture 1294′ of stent 1219′. When these control wires 1280/1281 are withdrawn distally by operation of a handle of the delivery system 1270, the respective end of stent graft 1211 is no longer constrained, and can expand radially when the operator has determined that the implant is in a position amenable to proper sealing and fixation.

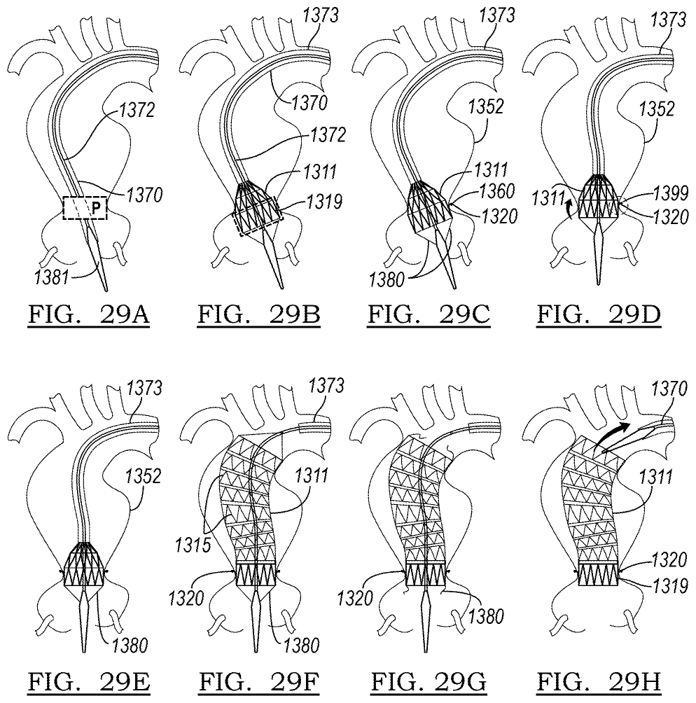

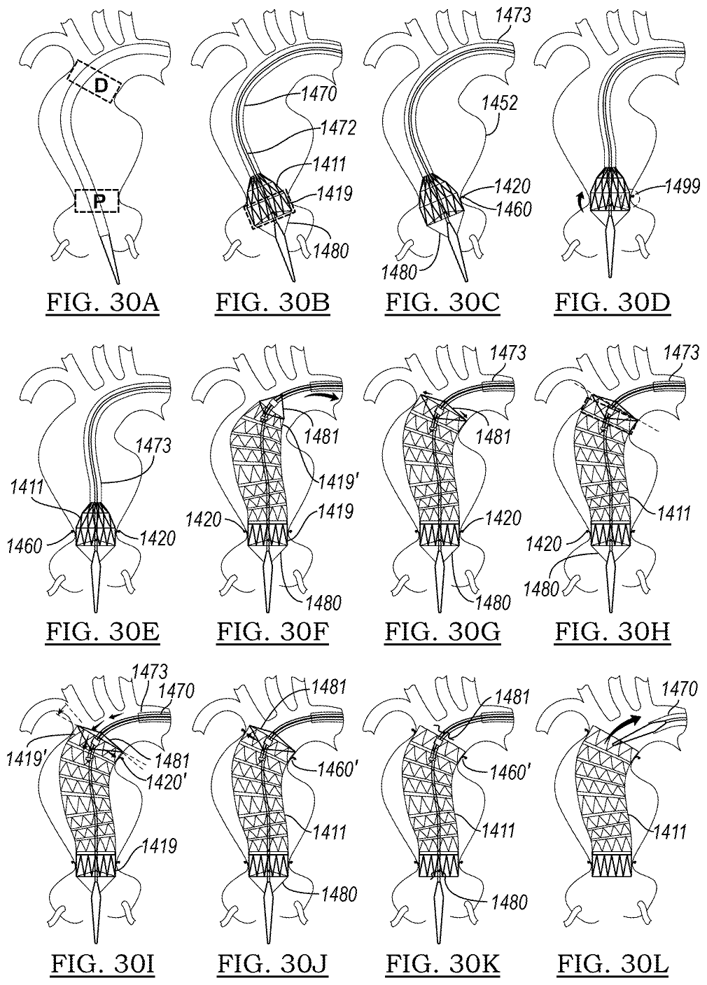

In one embodiment, a delivery system according to the principles of the present disclosure may include a stent graft having a proximal stent with controllable anchors, or a distal stent ring with controllable anchors, or both. FIGS. 29A-29H illustrate steps in a deployment process of a stent graft 1311 having a proximal stent ring 1319 having a set of controllable anchors 1320; FIGS. 30A-30L likewise illustrate steps in a deployment process of a stent graft 611 having a proximal stent ring 1419 having a set of controllable anchors 1420 and a distal stent ring 1419′ having a set of controllable anchors 1420′.

In a first step shown in FIG. 29A, a delivery assembly 1370 is led via tip 1381 into the region of an aneurysm of vessel 1350. Ideal proximal landing zone P for the stent graft 1311 is shown as a rectangle at a thinner, relatively more healthy region of the vessel near the aneurysm. In FIG. 29A, the outer sheath 1373 has been retracted distally by an operator, allowing a portion of the stent graft 1311 to emerge therefrom and expand. As can be seen in FIG. 29B, the proximal stent ring 1319 has landed at an angle relative to proximal landing zone P. By using a visualization technique, the operator may discern whether at least a portion of the device is disposed in such a manner that implantation can move forward.

In FIG. 29C, it has been determined that an anchor 1320 is in position such that the stent 1311 can be implanted, and so the anchor control wire 1357 (not shown) that controls the anchor 1320 is pulled distally, and the anchor 1320 adopts its expanded configuration 1360. As shown in FIG. 29D, securely implanted anchor 1320 may be used as a pivot 1399, the delivery assembly 1370 being shifted such that the stent graft 1311 pivots around the point of the vessel 1352 until it lands substantially in ideal proximal landing zone P as defined in FIG. 29A.

In FIG. 29E, the remaining anchors 1320 of the proximal stent 1319 are expanded to their expanded configurations 1360, and in FIG. 29F, the outer sheath 1373 is withdrawn distally to allow the remainder of stent graft 1311 it expand into the body lumen. In FIG. 29G, the control wires 1380/1381 are released, and in FIG. 29H, the pusher 1372 is resheathed and assembly 1370 is withdrawn, leaving stent graft 1311 implanted in the lumen of the vessel 1352 to be treated.

FIGS. 30A-30L depict a method of delivery of a stent graft 1411 similar to that illustrated in FIGS. 29A-29H, but using both a proximal stent ring 1419 having controllable anchors 1420, and a distal stent ring 1419′ having controllable anchors 1420′. As can be seen in FIG. 30A, both an ideal proximal landing zone P and an ideal distal landing zone D are illustrated at or near the necks of an aneurysm in vessel 1452. Steps in FIGS. 30B-30E are similar to the steps of FIG. 29B-29E, including using a deployed anchor 1420 as a pivot 1499 for better landing of the stent graft 1411 in FIG. 30D.

In FIG. 30F, the outer sheath 1473 is withdrawn distally in order to more completely release the stent graft 1411 from delivery assembly 1470. The control wires 1481 constrain the distal end of stent graft 1411, particularly distal stent ring 1419′, from the fully radially expanded configuration. In FIG. 30G, the stent control wires 1481 are withdrawn distally, slackening the wires, and allowing the distal stent ring 1419′ to expand radially. As shown in FIG. 30H, the distal stent ring 1419′ has landed in a position that differs from ideal landing zone D of FIG. 30A. Therefore, one anchor 1420′ which is in the proper position, as can be seen in FIG. 30H, is released by withdrawing an anchor control wire distally, allowing the anchor 1420 to adopt its expanded configuration 1460′ and engage the vessel wall. Similarly to the proximal stent ring 1419, the distal stent ring 1419′ is pivoted around the deployed anchor 1420′ and moved more closely to meet ideal landing zone D in FIG. 30I. Thereafter, as shown in FIG. 30J, the remaining controllable anchors 1420′ are released to adopt their expanded configurations 1460′, and control wires 1481 are further withdrawn, as shown in FIG. 30K. Finally, in FIG. 30L, the pusher 1472 is resheathed and the delivery assembly 1470 is removed from the vasculature of the patient and the procedure is completed.

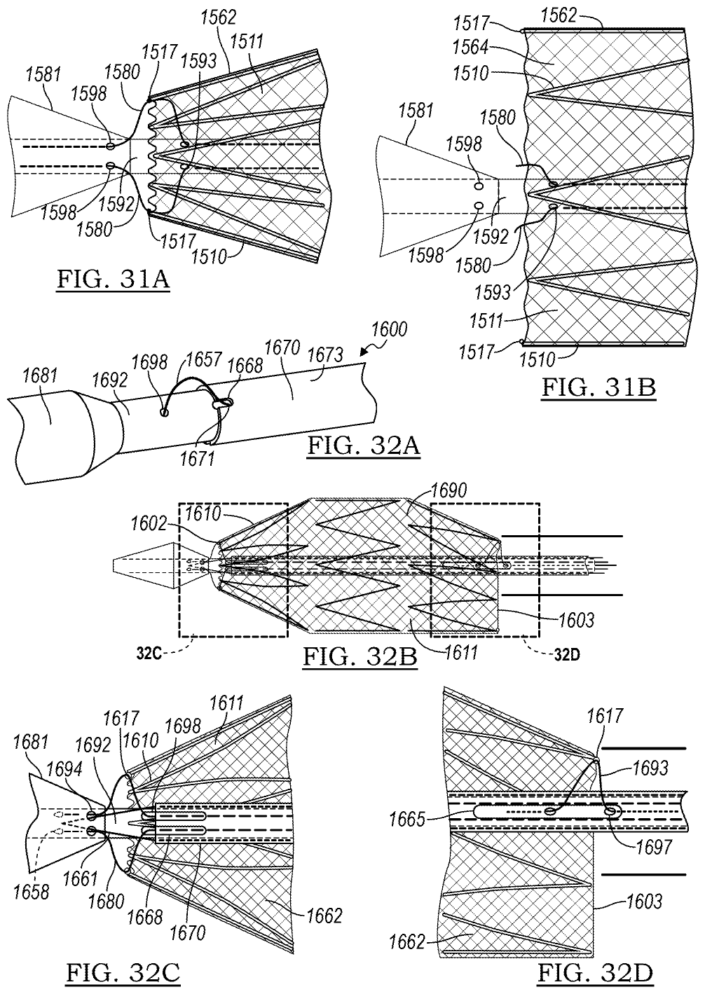

FIG. 31A shows a close-up of a stent graft 1511 of a conventional design in which stent control wires 1557 are fed proximally out through apertures 1593 of tubular member 1592, through stent apertures 1517 at the proximal end of stent graft 1511, and back into tubular member 1592 via proximal ports 1598 of the delivery assembly 1570. The apertures 1592/1593 are small, circular ports, sized to just allow the wires 1557 to be threaded through. The wires 1557 play a dual role of device fixation and release. As shown in FIG. 31B, withdrawal of the wires causes a sudden opening of the proximal end of the stent graft 1511. The stored elastic energy from the compressed stent graft 1511 is released upon opening, which may cause a “jump,” leading to an unintended orientation of the implant in the vessel

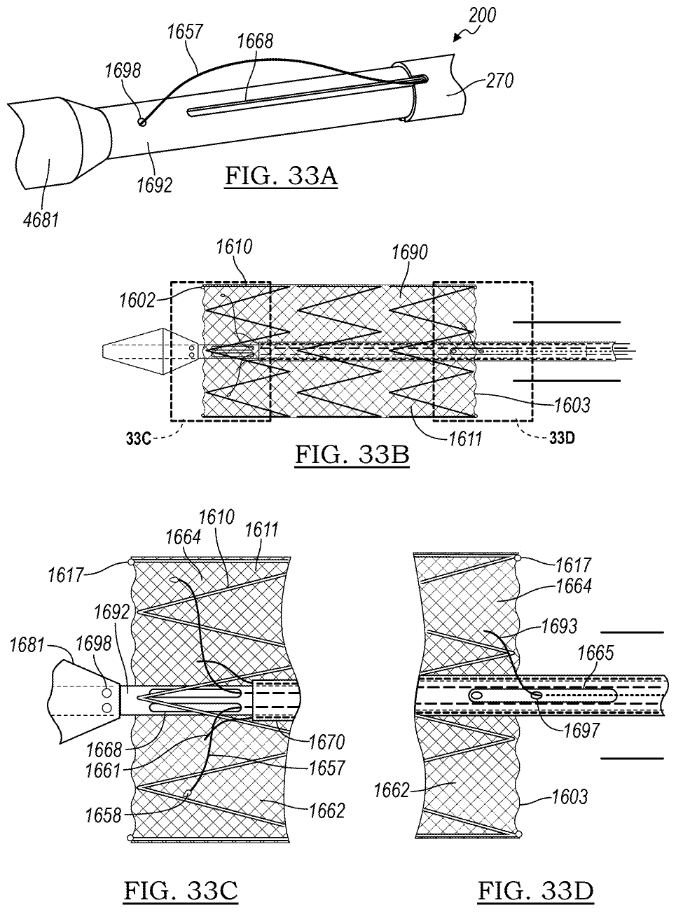

A way of providing control during this step is illustrated in FIGS. 32A-32D and 33A-33D. Elongate slot 1668 in tubular member 1692 allows for more controlled deployment of the end of a medical device, including a stent graft. As shown in FIG. 32A, a proximal aperture 1698 may still be provided as a rounded hole for wire 1657, but the distal aperture takes on the form of an elongate slot 1668, which has a length greater than the diameter of a circular aperture would be. For simplicity, only a single wire 1657 is depicted, but embodiments will be illustrated later that have multiple wires 1657 for control of multiple points of the device 1611.

Tubular member 1692 may be a cannula, such as a nitinol cannula, for use in delivery of a stent graft. Outer tubular member 1673 may be a nylon “skin” that may be withdrawn distally toward the operator, and may optionally include a notch 1671 that meets the slot 1668 and permits extension of the wire 1657 out therethrough. FIG. 33A illustrates the device 1600 of FIG. 32A, when the outer skin 1673 has been withdrawn proximally, exposing the elongate slot 1668 and allowing the wire 1657 to slacken.

FIGS. 32B-32D illustrate a delivery system including a stent graft 1611, wherein the proximal stent 1619 is held in a collapsed or delivery configuration. When the slideable outer member 1673 is removed, the intrinsic tension in the control wire 1657 is released, and the wire 1657 is free to move and the radial expansion force from the proximal end of proximal stent 1619 of the stent graft 1611 overrides the control wires 1657, allowing radial expansion of the stent graft 1611. Optionally, chocking wires 1661 may be included in order to provide a method of ensuring that control wires 1657 do not disengage prematurely. The chocking wires 1661 may be independently controllable and may be attached at their ends to control wires 1657, or may simply be fed through the same aperture (such as aperture 1698) to keep the control wire 1657 in place until such time as the chocking wire 1661 is withdrawn.

Once the slidable cannula skin 1673 has been moved distally to unlock the wires as in FIGS. 33B-33D, the user can manually control the tension of the wires, against the radial force of the proximal stent 1619, to manipulate the radius of the proximal stent 1619 remotely. The user can therefore simulate how the proximal stent 1619 will orient during landing before committing to a final deployed state, thereby allowing improved positioning.

A delivery assembly 1670 having an elongate slot 1668 for a retention wire 1657 may provide sufficient control over the deployment position of a medical device that controllable anchors may be an optional feature of said device. A person of skill in the art may make a determination about whether a particular application calls for controllable anchors.

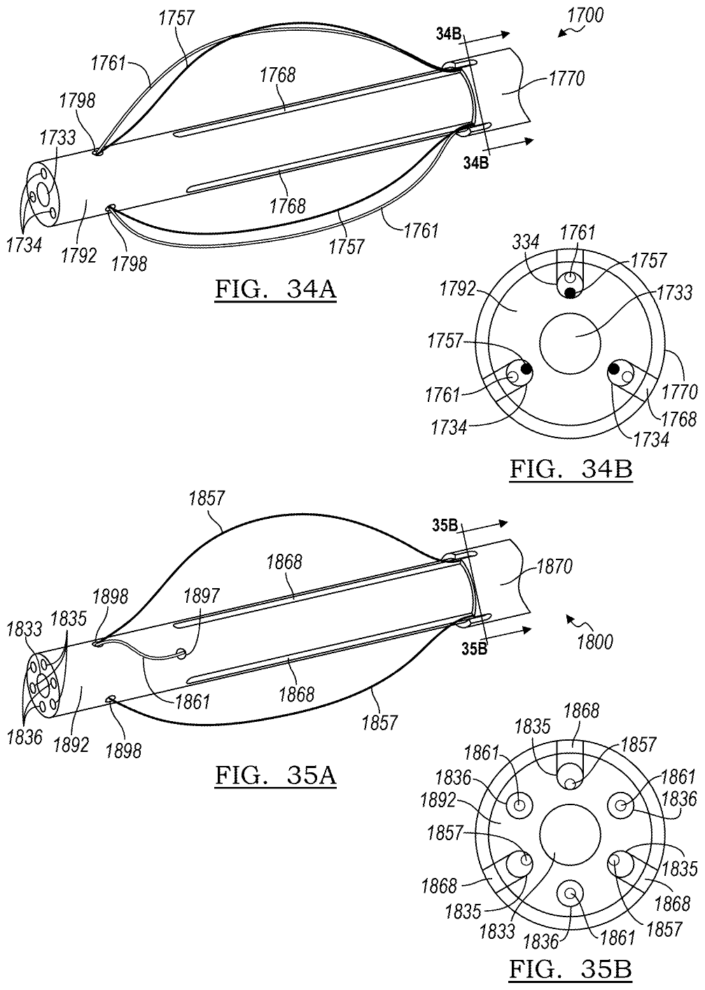

FIGS. 34A-34B and 35A-35B illustrate embodiments of delivery assemblies 1770/1870 having multiple lumens for multiple control wires.

The embodiment of FIGS. 34A-34B features a tubular member 1792 having a central lumen 1733 and a plurality of wire lumens 1734. The depicted embodiment includes three wire lumens 1734, but more or fewer may be employed, depending on the device to be delivered. As can best be seen in the cross-sectional view of FIG. 34B, the wire lumens 1734 are shared lumens, containing both retention wires 1757 and chocking wires 1761. These wires are controllable independent of one another, despite sharing wire lumens 1734. As shown in cross section of FIG. 34B, the wire lumens 1734 are in fluid communication with the elongated slots 1768, allowing the wires to emerge from the tubular member 1792.

In the embodiment of FIGS. 35A-35B, the assembly 1870 includes multiple types of wire lumens; three control wire lumens 1835, housing control wires 1857, surround central lumen 1833, disposed in alternating fashion with chocking wire lumens 1861. In this design, the control wire lumens 1835 are in fluid communication with the elongate slots 1868, while the chocking wires 1861 emerge through chocking wire apertures 1897. The chocking wires 1861 and the control wires 1857 converge and make contact at or near apertures 1898, where they re-enter the interior of tubular member 1892.

In other embodiments, such as those for use in delivery systems that include a medical device featuring controllable anchors, the shared lumens 1734 of the device 1770 of FIGS. 34A-34B may be shared between stent control wires and anchor control wires, and for example, the device of FIGS. 35A-35B may have anchor control wire lumens instead of, or in addition to, lumens housing chocking wires.

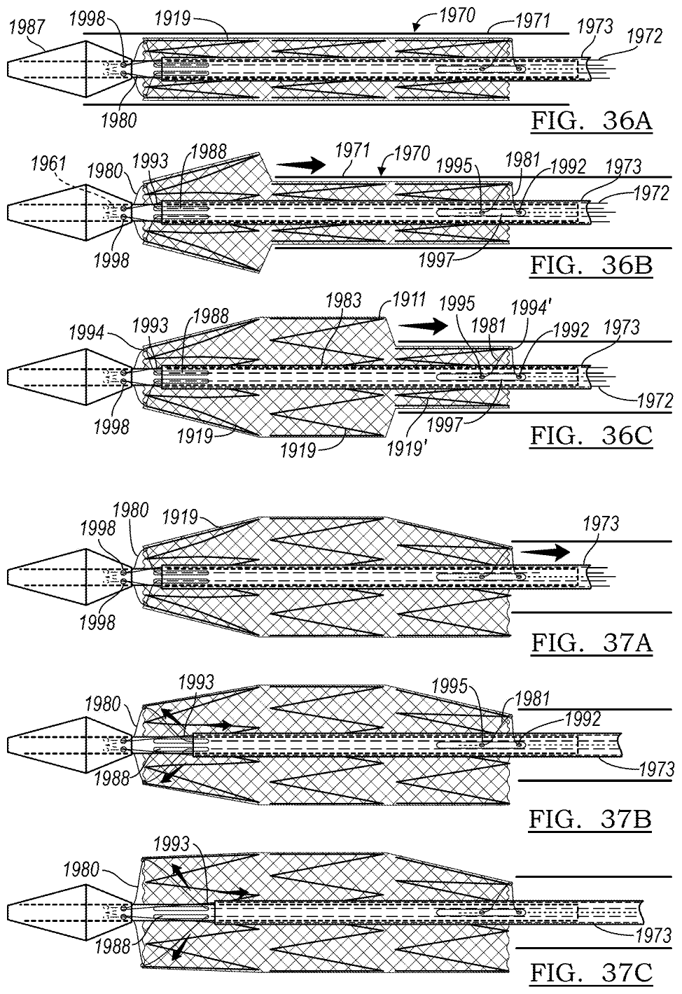

FIGS. 36A-36C, 37A-37C, and 38A-38F illustrate steps in the delivery and expansion of a stent graft 1911 by a delivery assembly 1970. For simplicity, the body vessel or lumen is not shown. The stent graft 1911 has a proximal stent ring 1919 which is self-expanding and which is tethered to the delivery assembly 1970 by stent control wires 1980. The stent control wires 1980 are threaded through apertures 1994 at the proximal end of stent ring 1919. Likewise, self-expanding distal stent 1919′ is restrained by control wires 1981 threaded through apertures 1994′ at the distal end of stent 1919′. First tubular member 1972 is surrounded in part by second tubular member 1973. The stent graft 1911 is shown in its collapsed, or delivery, configuration in FIG. 36A.

In FIG. 36B, the outer sheath 1971 is withdrawn distally by the operator to allow proximal stent ring 1919 to partially expand. The stent ring 1919 is still partially constrained by control wires 1980, which are in turn constrained by chocking wires 1961. The outer sheath 1971 is further withdrawn in FIG. 36C, allowing more of the stent graft 1911 to expand radially.

In FIG. 37A, the second tubular member 1973 is withdrawn distally by the operator to reveal more of elongated slots 1968. This, in turn, introduces slack into control wires 1980, allowing for controlled radial expansion of proximal stent ring 1919. In FIG. 37B, the control wires 1980 have slackened further, allowing the proximal end of stent graft 1911 to open radially, and in FIG. 37C, the control wires 1980 have slackened yet further, allowing the stent ring 1919 to reach a nearly fully radially expanded state.

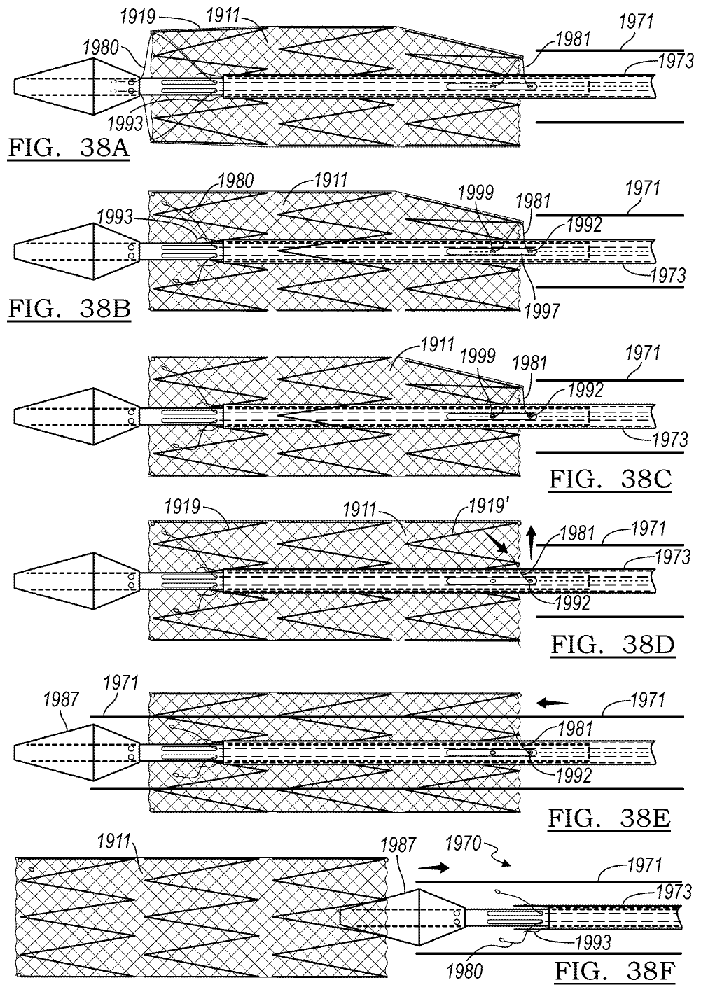

In the step shown in FIG. 38A, the outer sheath 1971 is fully withdrawn and lies distal the distal end of stent graft 1911. The distal end has partially expanded radially due to the self-expanding nature of stent ring 1919′, but the distal control wires 1981 continue to partially radially constrain this end of the stent graft 1911. The control wire 1981 may exit aperture 1992 of first tubular member 1972, thread through aperture 1994′ of stent ring 1919′, and back through aperture 1995 into a wire lumen of first tubular member 1972. The apertures 1992 and 1995 may optionally be provided in a depression 1997 of the outer surface of first tubular member 1972.

In FIGS. 38B and 38C, the chocking wires 1993 are disconnected from control wires 780 by the operator, allowing proximal stent ring 1919 to assume its fully expanded condition. In FIG. 38D, control wire 1981 is withdrawn distally, allowing further radial expansion of distal stent ring 1919′.

In FIG. 38E, the control wire 1981 no longer engages stent graft 1911, allowing distal stent ring 1919′ to fully radially expand. Outer sheath 1971 is advanced proximally to join the remainder of delivery assembly 1970, and in the step of FIG. 38F, the assembly 1970 is withdrawn from the vasculature of the patient. Thus, the implant (stent graft 1911) is deployed to the body lumen.

### CLAUSES

1. A medical device for implantation into a body lumen, the medical device being movable between a delivery configuration and an expanded configuration, the medical device comprising: an anchor comprising an elongate ribbon extending from a first end to a second end and a longitudinally expandable section defining at least one curve, the first end of the elongate ribbon being connected to the longitudinally expandable section, the elongate ribbon comprising a barb formed therefrom, wherein the medical device defines a lumen, the anchor being disposed within the lumen in the delivery configuration, and wherein the anchor extends outward radially from the lumen in the expanded configuration.

2. The medical device of clause 1, wherein the medical device comprises a stent.

3. The medical device of clause 2, wherein the stent is a cannula-cut stent.

4. The medical device of clause 1, wherein the medical device comprises a body which is substantially tubular and defines a longitudinal axis therethrough.

5. The medical device of clause 4, wherein the longitudinally expandable section defines a plurality of curves along a circumferential portion of the body.

6. The medical device of clause 1, wherein the barb is disposed within the elongate ribbon in the delivery configuration.

7. The medical device of clause 1, wherein the barb extends radially outward from the elongate ribbon in the expanded configuration.

8. The medical device of clause 1, wherein the anchor further comprises a radiopaque marker.

9. The medical device of clause 1, wherein the anchor terminates in a hook.

10. The medical device of clause 9, wherein the radiopaque marker is disposed on the barb.

11. The medical device of clause 2, wherein the stent comprises a plurality of struts, at least one anchor being formed within one of the plurality of struts.

12. The medical device of clause 1, wherein the medical device comprises a shape memory material, the medical device being biased to adopt the first configuration.

13. The medical device of clause 1, wherein the second end of the barb lacks a hook, and the anchor is retractable during deployment.

14. A stent for implantation into a body lumen, the stent comprising a tubular body defining a lumen therein and longitudinal axis therethrough, the stent being movable between a delivery configuration and an expanded configuration, the stent comprising: an anchor comprising an elongate ribbon extending from a first end to a second end and a longitudinally expandable section defining at least one curve, the first end of the elongate ribbon being connected to the longitudinally expandable section wherein the anchor is disposed within the lumen when the stent is in the delivery configuration, and wherein the anchor extends outward radially from the lumen in the expanded configuration.

15. The stent of clause 14, wherein the longitudinally expandable section defines a plurality of curves along a circumferential portion of the tubular body.

16. The stent of clause 14, comprising a plurality of struts, at least one anchor being formed within one of the plurality of struts.

17. The stent of clause 14, wherein the second end of the barb comprises a hook.

18. The stent of clause 14, wherein the anchor further comprises a radiopaque marker.

19. The stent of clause 14, wherein the medical device comprises a shape memory material, the medical device being biased to adopt the first configuration.

20. A method of making a stent, comprising: cutting a shape memory metal cannula to define a stent body, the stent body being tubular and defining a lumen therethrough, the stent body comprising at least one anchor for contacting tissue, the anchor comprising a longitudinally expandable section and an elongate ribbon extending from a first end to a second end, the first end of the elongate ribbon being connected to the longitudinally expandable section, the elongate ribbon comprising a barb, placing the stent body in a mandrel, the mandrel defining a first curved surface and a second curved surface, such that the anchor is disposed between the first curved surface and the second curved surface, and within an interior of the stent body, and heating the stent body to a temperature effective to define a remembered state wherein the anchor is disposed within the interior of the stent body.

20A. A medical device system comprising: a medical device movable between a delivery configuration and an expanded configuration, the medical device comprising: an anchor comprising an elongate ribbon extending from a first end to a second end and a longitudinally expandable section defining at least one curve, the first end of the elongate ribbon being connected to the longitudinally expandable section, the elongate ribbon comprising a barb formed therefrom, wherein the medical device comprises at least one aperture for receiving a release wire therethrough, and wherein the medical device defines an outer perimeter, the anchor being restrained within the outer perimeter by the release wire in the delivery configuration, and the anchor extending outward radially from the outer perimeter in the expanded configuration; and the release wire for retaining the medical device in the delivery configuration.

21. A delivery system for delivering a medical device, the delivery system comprising: a first tubular member comprising a wall and extending from a proximal end to a distal end and defining a longitudinal axis therethrough, the first tubular member comprising at least one wire lumen defined therethrough, the at least one wire lumen being substantially parallel to the longitudinal axis, the wall in a distal portion of the first tubular member having at least one elongate aperture formed therethrough such that each wire lumen is accessible through an elongate aperture; a retention wire disposed in the at least one wire lumen; and the medical device having a delivery configuration and a deployed configuration, the medical device defining a generally tubular outer perimeter in the delivery configuration and being disposed about the first tubular member, the medical device comprising at least one anchor which is extends radially beyond the outer perimeter in the deployed configuration, the anchor defining at least one aperture for receiving the retention wire.

22. The delivery system of clause 21 comprising a second tubular member extending from a proximal end to a distal end and being disposed about the first tubular member.

23. The delivery system of clause 21 comprising at least one chocking wire, each retention wire being releasably connected to a chocking wire.

24. The delivery system of clause 23, wherein the retention wire is directly connected to the chocking wire.

25. The delivery system of clause 23, comprising a plurality of wire lumens.

26. The delivery system of clause 25, wherein each of the plurality of wire lumens includes a retention wire and a chocking wire therein.

27. The delivery system of clause 25, wherein the plurality of wire lumens comprises a plurality of first lumens each comprising a retention wire, and a plurality of second lumens each comprising a chocking wire.

28. The delivery system of clause 27, wherein the plurality of first lumens and the plurality of second lumens are arranged in a circular fashion through the first tubular member, such that each first lumen is located between two second lumens in a circumferential direction.

29. The delivery system of clause 27, wherein the wall of the first tubular member includes a plurality of apertures therethrough, each aperture being in fluid communication with a second lumen.

30. The delivery system of clause 22, wherein the second tubular member defines a plurality of slots at the distal end, each slot being configured to align with an elongate aperture.

31. The delivery system of clause 21, wherein each retention wire is connected to a portion of a stent graft to be delivered.

32. A delivery system for delivering a medical device, the delivery system comprising: a first tubular member comprising a wall and extending from a proximal end to a distal end and defining a longitudinal axis therethrough, the first tubular member comprising a plurality of wire lumens defined therethrough, each of the plurality of wire lumens being substantially parallel to the longitudinal axis, the wall in a distal portion of the first tubular member having at least one elongate aperture formed therethrough such that each wire lumen is accessible through an elongate aperture; a retention wire disposed in each of the plurality of wire lumens; a chocking wire disposed in each of the plurality of wire lumens and releasably connected to the retention wire disposed in the respective wire lumen; and a stent graft to be delivered, the stent graft including at least one aperture for engagement of at least one retention wire.

33. The delivery system of clause 32, comprising a second tubular member extending from a proximal end to a distal end and being disposed about the first tubular member.

34. The delivery system of clause 32, wherein each retention wire is directly connected to the respective chocking wire.

35. The delivery system of clause 32, wherein the second tubular member defines a plurality of slots at the distal end, each slot being configured to align with an elongate aperture.

36. A stent graft for implantation into a body lumen, the stent graft comprising a tubular body defining a lumen therethrough and a longitudinal axis, the stent graft being movable between a delivery configuration and an expanded configuration, the stent graft comprising: a plurality of stent rings, at least one of the plurality of the stent rings comprising an anchor comprising an elongate ribbon extending from a first end to a second end and a longitudinally expandable section, the first end of the elongate ribbon being connected to the longitudinally expandable section, the elongate ribbon comprising a barb formed therefrom, wherein the anchor comprises at least one aperture for receiving a release wire therethrough, and wherein the stent graft defines an outer perimeter, the anchor being restrained within the outer perimeter by the release wire in the delivery configuration, the anchor extending outward radially from the outer perimeter in the expanded configuration.

37. The stent graft of clause 36, wherein the at least one stent ring comprising an anchor is a proximal stent ring.

38. The stent graft of clause 36, wherein the at least one stent ring comprising an anchor is a distal stent ring.

39. The stent graft of clause 36, comprising a proximal stent ring and a distal stent ring, both the proximal stent ring and the distal stent ring comprising at least one anchor each.

40. The stent graft of clause 36, wherein the at least one stent ring is a self-expanding stent ring.

As a person skilled in the art will readily appreciate, the above description is only meant as an illustration of implementation of the principles this application. This description is not intended to limit the scope of this application in that the system is susceptible to modification, variation and change, without departing from the spirit of this application, as defined in the following claims.

What is claimed is:

1. A medical device for implantation into a body lumen, the medical device being movable between a delivery configuration and an expanded configuration, the medical device comprising:
an anchor comprising an elongate ribbon extending from a first end to a second end and a longitudinally expandable section defining at least one curve, the first end of the elongate ribbon being connected to the longitudinally expandable section, the elongate ribbon comprising a barb formed therefrom,
wherein the medical device comprises at least one aperture for receiving a release wire therethrough, and
wherein the medical device defines an outer perimeter, the anchor being restrained within the outer perimeter by the release wire in the delivery configuration, and the anchor extending outward radially from the outer perimeter in the expanded configuration.

2. The medical device of claim 1, wherein the medical device comprises a stent.

3. The medical device of claim 2, wherein the stent is a cannula-cut stent.

4. The medical device of claim 1, wherein the medical device comprises a tubular body defining a longitudinal axis therethrough, the elongate ribbon being disposed substantially parallel to the longitudinal axis.

5. The medical device of claim 1, wherein the anchor defines a first aperture therethrough for receiving the release wire proximate the first end, and defines a second aperture therethrough for receiving the release wire proximate the second end.

6. The medical device of claim 1, wherein the barb terminates in a hook.

7. The medical device of claim 1, wherein the barb is disposed within the elongate ribbon in the first configuration.

8. The medical device of claim 1, wherein the barb extends radially outward from the elongate ribbon in the second configuration.

9. The medical device of claim 1, wherein the anchor further comprises a radiopaque marker.

10. The medical device of claim 9, wherein the radiopaque marker is disposed on the barb.

11. The medical device of claim 2, wherein the stent comprises a plurality of struts, at least one anchor being formed within one of the plurality of struts.

12. The medical device of claim 1, wherein the medical device comprises a shape memory material, the medical device being biased to adopt the second configuration.

13. The medical device of claim 1, wherein the second end of the barb lacks a hook, the anchor being retractable during deployment.

14. A stent for implantation into a body lumen, the stent comprising a tubular body defining a lumen therethrough and a longitudinal axis, the stent being movable between a delivery configuration and an expanded configuration, the stent comprising:
an anchor comprising an elongate ribbon extending from a first end to a second end and a longitudinally expandable section, the first end of the elongate ribbon being connected to the longitudinally expandable section, the elongate ribbon comprising a barb formed therefrom,
wherein the stent comprises at least one aperture for receiving a release wire therethrough, and
wherein the stent defines an outer perimeter, the anchor being restrained within the outer perimeter by the release wire in the delivery configuration, the anchor extending outward radially from the outer perimeter in the expanded configuration.

15. The stent of claim 14, wherein the longitudinally expandable section defines at least one curve along a circumferential portion of the tubular body.

16. The stent of claim 14, comprising a plurality of struts, at least one anchor being formed within one of the plurality of struts.

17. The stent of claim 14, wherein the second end of the barb comprises a hook.

18. The stent of claim 14, wherein the anchor further comprises a radiopaque marker.

19. The stent of claim 14, wherein the medical device comprises a shape memory material, the medical device being biased to adopt the first configuration.

20. A stent graft for implantation into a body lumen, the stent graft comprising a tubular body defining a lumen therethrough and a longitudinal axis, the stent graft being movable between a delivery configuration and an expanded configuration, the stent graft comprising:
a plurality of stent rings, at least one of the plurality of the stent rings comprising an anchor comprising an elongate ribbon extending from a first end to a second end and a longitudinally expandable section, the first end of the elongate ribbon being connected to the longitudinally expandable section, the elongate ribbon comprising a barb formed therefrom,
wherein the anchor comprises at least one aperture for receiving a release wire therethrough, and
wherein the stent graft defines an outer perimeter, the anchor being restrained within the outer perimeter by the release wire in the delivery configuration, the anchor extending outward radially from the outer perimeter in the expanded configuration.
